(19) United States
(12) Patent Application Publication
(10) Pub. No.: US 2024/0207186 A1
(43) Pub. Date: Jun. 27, 2024

(54) NANOLIPOPROTEIN PARTICLES AND RELATED COMPOSITIONS METHODS AND SYSTEMS FOR LOADING RNA

(71) Applicant: LAWRENCE LIVERMORE NATIONAL SECURITY, LLC, Livermore, CA (US)

(72) Inventors: Kurt I. KAMRUD, San Diego, CA (US); Nathaniel S. WANG, San Diego, CA (US); Martina FELDERMAN, La Jolla, CA (US); Bolyn HUBBY, Redwood City, CA (US); Heather D, GOUVIS, San Diego, CA (US); Nicholas O. FISCHER, Livermore, CA (US); Matthew A. COLEMAN, Oakland, CA (US); Angela Clare EVANS, Livermore, CA (US); Wei HE, Davis, CA (US); Amy RASLEY, Livermore, CA (US); Craig D. BLANCHETTE, San Leandro, CA (US)

(21) Appl. No.: 18/422,810

(22) Filed: Jan. 25, 2024

Related U.S. Application Data
(62) Division of application No. 15/969,311, filed on May 2, 2018.
(60) Provisional application No. 62/500,433, filed on May 2, 2017.

(51) Int. Cl. A61K 9/127; A61K 47/69; A61K 48/00; C12N 15/88

(52) U.S. Cl. CPC A61K 9/1272; A61K 9/1274; A61K 47/6917; C12N 15/88; A61K 48/0008

(57) ABSTRACT

Nanolipoprotein particles comprising at least a scaffold protein component and a membrane lipid component and related complexes, compositions, methods and systems are described, in which the membrane lipid component comprises at least one or more membrane forming lipids and one or more cationic lipids.

### CROSS-REFERENCE TO RELATED APPLICATIONS

This application claims priority under 35 USC § 119(e) to U.S. Application Ser. No. 62/500,433 filed May 2, 2017. The disclosure of the prior application is considered part of and is incorporated by reference in the disclosure of this application in its entirety.

### STATEMENT OF GOVERNMENT GRANT

The invention was made with Government support under Contract No. DE-AC52-07NA27344 between the U.S. Department of Energy and Lawrence Livermore National Security, LLC, for the operation of Lawrence Livermore National Security. The Government has certain rights to the invention.

### FIELD

The present disclosure relates to nanolipoprotein particles (NLPs) and, in particular, nanolipoprotein particles incorporated with cationic lipid compounds and related compositions methods and systems.

### BACKGROUND

Nanolipoprotein particles are nanometer-sized particles usually comprised of an amphipathic lipid bilayer and an apolipoprotein. NLPs have been used for various biotechnology applications, such as membrane protein stabilization/solubilization, drug delivery, and in particular vaccine delivery, and diagnostic imaging.

In some instances, NLPs can self-assemble under appropriate conditions into nano-scale amphipathic apolipoprotein-stabilized lipid bilayer particles possibly comprising additional lipid or protein molecules inserted into or attached to the amphipathic component of the NLP. The self-assembled particles are typically formed by an apolipoprotein encircling a nanometer scale lipid bilayer defining a nanolipoprotein particle.

Despite the advancement of this technology, providing NLPs including desired functionalities and/or with a desired stability can be challenging.

### SUMMARY

Provided herein are nanolipoprotein particles, and related compositions, methods and systems, which comprise one or more membrane forming lipids, one or more cationic lipids and a scaffold protein. In several embodiments, nanolipoprotein particles herein described can be used as a delivery vehicle to deliver in vitro or in vivo large nucleic acids complex with improved transfection efficiency.

According to a first aspect, a cationic nanolipoprotein particle is described. The cationic nanolipoprotein particle comprises one or more membrane forming lipids, one or more cationic lipids and one or more scaffold proteins, the membrane forming lipid and the cationic lipids arranged in a discoidal membrane lipid bilayer stabilized by the scaffold protein.

According to a second aspect, a cationic-NLPs-polynucleotide complex is described. The cationic-NLPs-polynucleotide complex comprises a polynucleotide molecule attached to one or more cationic NLPs wherein each cationic NLP comprising one or more cationic lipids and a membrane forming lipid arranged in a discoidal membrane lipid bilayer stabilized by a scaffold protein. In particular, in some embodiments, cationic-NLPs-polynucleotide complex polynucleotides and NLPs are included in a ratios polynucleotide:NLPs of 2:1 or higher.

According to a third aspect, a cationic-NLPs-polynucleotide complex is described. The cationic-NLPs-polynucleotide complex comprises a polynucleotide molecule, in particular a polynucleotide having at least 200 bases, from 200 to 5000 bases, at least 5,000 bases, from 5,000 to 15,000 bases and higher than 15,000 bases attached to a plurality of cationic NLPs each cationic NLP comprising one or more cationic lipids and a membrane forming lipid arranged in a discoidal membrane lipid bilayer stabilized by a scaffold protein.

According to a fourth aspect, a method and system to provide a polynucleotide-nanolipoprotein complex is described. The method comprises contacting the polynucleotide with a plurality of cationic nanolipoprotein particles each comprising a membrane lipid bilayer stabilized by a scaffold protein in a discoidal configuration with the membrane lipid bilayer comprising one or more cationic lipids, to provide a polynucleotide cationic-nanolipoprotein-complex comprising the polynucleotide attached to the plurality of cationic nanolipoprotein particles.

The system comprises one or more polynucleotide molecules, one or more cationic nanolipoprotein particles comprising one or more cationic lipids and one or more membrane forming lipids within a membrane lipid bilayer stabilized by a scaffold protein. In the system, the one or more polynucleotide and the one or more cationic nanolipoprotein particles provides are for simultaneous combined or sequential use in the method to provide a polynucleotide nanolipoprotein complex herein described.

According to a fifth aspect, a method and system to provide a cationic nanolipoprotein particle, are described. The method comprises contacting a membrane forming lipid and one or more cationic lipids with a scaffold protein to provide a discoidal lipid bilayer comprising the membrane forming lipid and the one or more cationic lipids stabilized by the scaffold protein.

The system comprises one or more membrane-forming lipids, one or more cationic lipids, and a scaffold protein. In the system, assembly of the one or more membrane forming lipids and the scaffold protein provides a nanolipoprotein particle in which the one or more cationic lipids are comprised within a membrane lipid bilayer stabilized by the scaffold protein.

According to a sixth aspect, a composition is described comprising one or more cationic nanolipoprotein particles and/or one or more cationic-NLPs-polynucleotide complexes, together with an acceptable vehicle. In some embodiments, the composition can be a pharmaceutical composition and the acceptable vehicle can be a pharmaceutically acceptable vehicle. In some of those embodiments, the composition can be a vaccine.

According to a seventh aspect, a method and system to deliver one or more polynucleotide molecules to target environment is described. The method comprises contacting the target environment with one or more cationic-NLPs-polynucleotide complexes herein described comprising the one or more polynucleotide molecules.

The system comprises one or more cationic nanolipoprotein particles herein described and one or more polynucleotide molecules for combined use in the method to deliver one or more polynucleotide molecules herein described.

According to additional aspects, methods and systems, comprising forming and using the cationic nanolipoprotein particles herein described are also provided in the present disclosure. Methods and systems to perform an assay on nucleotide molecules loaded in the cationic-NLP-nucleotide complexes of the present disclosure are also described.

Cationic nanolipoprotein particles and related complexes, compositions, methods and systems, in several embodiments provide an efficient, versatile and reproducible delivery of RNA replicon molecules. In particular, the cationic NLP-replicon complex formulations are stable, easily prepared and do not fully encapsulate the replicon while they can provide protection against RNase degradation, shielding access to outside molecules as well as increasing the replicon's in vivo transfection efficiency.

Cationic nanolipoprotein particles and related complexes, compositions, methods and systems, in several embodiments show in several embodiments an ability to load RNA replicon molecules having a 10,000 bp or higher.

Cationic nanolipoprotein particles and related complexes, compositions, methods and systems, in several embodiments allow increased transfection of an RNA replicon with respect to unformulated replicon through in vivo gene expression in a mouse model.

Cationic nanolipoprotein particles and related complexes, compositions, methods and systems, in several embodiments can be used to deliver the RNA with a ratio between the number of nitrogen atoms in the cationic NLPs and the number of phosphate groups in the RNA, 10-fold lower compared to conventional approaches.

The nanolipoprotein particles and related compositions, methods and systems herein described can be used in connection with various applications wherein highly efficient in vivo nucleic acids delivery and transfection is desired. For example, the nanolipoprotein particles herein described and related compositions methods and systems can be used as a vehicle for delivery of compounds such as therapeutics to a specific target destination, as a platform for immunostimulating agents, vaccine development and use, and/or to contain cell-targeting moieties. Additional exemplary applications include uses of nanolipoprotein particles in several fields including basic biology research, applied biology, bio-engineering, bio-energy, molecular biology, medical research, medical diagnostics, therapeutics, bio-fuels, and in additional fields identifiable by a skilled person upon reading of the present disclosure.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and example sections, serve to explain the principles and implementations of the disclosure. Exemplary embodiments of the present disclosure will become more fully understood from the detailed description and the accompanying drawings, wherein:

FIG. 1 shows a schematic illustration of exemplary cationic NLP (cationic-NLP) formation, wherein a scaffold protein of interest, bulk lipids, and cationic lipids are mixed with surfactant and incubated with biobeads overnight. The next morning, cationic-NLPs are filtered and tested by size exclusion chromatography (SEC) to check for proper homogeneity, peak distribution, and size of the cationic-NLP peak. The different steps are schematically indicated by arrows.

FIG. 2 shows an exemplary High Throughput Screen (HTS) approach that can be used to screen cationic-NLP formation in which different steps are schematically indicated by arrows.

FIG. 3 shows exemplary results of High Throughput SEC screening of NLPs. In the illustration of FIG. 3, the X-axis is SEC retention time (in minutes), the y-axis is absorbance intensity (in microvolts). NLPs were scored based on homogeneity of the NLP peak, the intensity of the free protein peak, as well as the amount of large aggregate species. The top SEC trace is an example of successful NLP formation, with scaffold protein apoA1, 80% DMPC bulk lipid, and 20% DMTAP cationic lipid. It received a score of 2. The bottom SEC trace shows poor NLP formation with scaffold protein apoA1, 80% DMPC bulk lipid, and 20% DOEPC cationic lipid. It received a score of 0.

FIG. 4 shows examples of SEC results illustrating NLP Stability. In the illustration of FIG. 4, the X-axis is SEC retention time (in minutes), the y-axis is absorbance intensity (in microvolts). 80:20 DMPC:DMTAP NLPs were injected onto SEC on day 1 to assess for proper NLP formation (results shown in bottom panel). After 50 days, the same sample was injected onto SEC after being stored at 4° C. (results shown in middle panel). An overlay of both results is shown in the top panel. NLPs shown are formed with apoA1 scaffold protein.

FIG. 5 shows diagrams illustrating exemplary results of size exclusion chromatography (SEC) performed on DMPC-containing cationic-NLPs comprising 1% DMTAP (black), 5% DMTAP (dashed), and 10% DMTAP (gray) (left panel) and on cationic-NLPs comprising 1% DOTAP (black), 5% DOTAP (dashed), and 10% DOTAP (gray) (right panel); in the illustration the detected assembly for each sample is shown in a score chart wherein full cationic-NLP assembly (white bar), cationic-NLPs assembly with margin loss in overall yield or homogeneity (light gray bar) and marginal cationic-NLP assembly with significant loss in yield or homogeneity (dark gray bar) are separately indicated.

FIG. 6 shows a diagram illustrating a score chart of results of High Throughput testing of exemplary cationic-NLPs using SEC analysis. Several bulk lipids (DMPC, POPC, or DOPC), scaffold proteins (ApoA1 or ApoE4) and cationic lipids were tested and assessed for proper cationic-NLP formation. Cationic-NLPs comprising cationic lipids DC-cholesterol (3β-[N—(N′,N′-dimethylaminoethane)-carbamoyl]cholesterol hydrochloride), DDAB (dimethyldioctadecylammonium (Bromide Salt)), DMEPC (1,2-dimyristoleoyl-sn-glycero-3-ethylphosphocholine), DMTAP (1,2-dimyristoyl-3-trimethylammonium-propane), DOEPC (1,2-dioleoyl-sn-glycero-3-ethylphosphocholine), DOTAP (1,2-dioleoyl-3-trimethylammonium-propane), or MVL5 (N1-[2-((1S)-1-[(3-aminopropyl)amino]-4-[di(3-amino-propyl)amino]butylcarboxamido)ethyl]-3,4-di[oleyloxy]-benzamide), at percentages of 1%, 5%, 10%, 15%, 20%, 30% or 40% were tested. The detected assembly for each sample is shown in a score chart wherein full cationic-NLP assembly was given a score of 2 (white bar), cationic-NLPs assembly with margin loss in overall yield or homogeneity was given a score of 1 (light gray bar), and marginal cationic-NLP assembly with significant loss in yield or homogeneity was given a score of 0 (dark gray bar), which are separately indicated. If the combination was not tested, it is left blank (diagonal line). This is a representative depiction of the cationic-NLPs tested.

FIG. 7 shows an exemplary illustration of the formation of cationic-NLP:Replicon complexes. NLPs are mixed with RNA Replicon at various NLP:RNA molar ratios. After simple mixing, the cationic lipids within the NLPs are attracted to the negatively charged RNA. Each RNA molecule binds to multiple NLPs to form a NLP:Replicon complex.

FIG. 8 shows images of gel electrophoresis results, in which relative migration of RNA based on varying NLP:RNA molar ratios are shown. Various bulk lipids (POPC, DOPC, or DMPC) are shown with several types of cationic lipids (DOTAP, DMTAP, or DDAB) at the percentages indicated. Each group shows increasing NLP:RNA molar ratios. NLP:RNA ratios are 15:1, 31:1, 62.5:1, 125:1, 250:1, and 500:1, respectively. All NLPs shown were formed with apoE4 scaffold protein. 0.1 μg RNA loaded in each well. RNA alone is shown as a control.

FIG. 9 shows images of gel electrophoresis results, in which relative migration of RNA with different cationic lipid percentages incorporated into NLPs are shown. Results for bulk lipid DMPC, or DMPC with cationic lipid MVL5 (1, 2, or 5%), or DMPC with cationic lipid DDAB (5, 10, or 20%) are shown. Each group shows increasing NLP:RNA molar ratios. NLP:RNA ratios are 15:1, 31:1, 62.5:1, 125:1, 250:1, and 500:1, respectively. Results are shown for NLP:RNA complexes either on day 1 or after 14 days stored at 4° C. All NLPs shown were formed with apoE4 scaffold protein. 0.1 μg RNA loaded per well. RNA alone is shown as a control.

FIG. 10 shows graphs of Zeta Potential distributions (measured in millivolts, mV) in some embodiments. Zeta potential was characterized for various cationic-NLP:RNA formulations at the ratios of NLP:RNA indicated in the X-axes. Results for bulk lipid DMPC, or DMPC with 2% cationic lipid MVL5, DMPC with 10% cationic lipid DDAB, DMPC with 20% cationic lipid DMTAP, or DMPC with 20% cationic lipid DMTAP and 20% glyceryl monooleate (GMO) additive are shown. RNA alone is shown as a control. Scaffold protein ApoE4 was used for DMPC alone, DMPC with 2% cationic lipid MVL5, and DMPC with 10% cationic lipid DDAB. Scaffold protein ApoA1 was used for DMPC with 20% cationic lipid DMTAP and DMPC with 20% cationic lipid DMTAP and 20% GMO additive.

FIG. 11 shows an exemplary zeta potential distribution for a cationic NLP:Replicon complex (98:2 DMPC:MVL5, left panels) and a non-cationic NLP:Replicon mixture (100% DMPC, right panels) at the ratios of NLP:RNA indicated. RNA alone is shown as a control. Scaffold protein ApoE4 was used for both the 100% DMPC example and the 98:2 DMPC:MVL5 example.

FIG. 12 shows a Table of results of the average size of replicon RNA alone, NLP alone with ApoE4 or ApoA1 scaffold proteins, and NLP:RNA complexes at the ratios indicated, assessed using Dynamic Light Scattering (DLS) analysis.

FIG. 13 shows a graph of results of Dye Exclusion analysis of NLP:Replicon ratio, measured in relative fluorescence units. Results are shown for NLP complexes comprising 100% DMPC (triangle), or NLP comprising DMPC with 10% DDAB (circle), or DMPC with 5% MVL5 (square). The ApoE4 scaffold protein was used for DMPC alone and with additional cationic lipids 5% MVL5, and 10% DDAB.

FIG. 14 shows images of gel electrophoresis results of migration patterns of RNA formulated with cationic-NLP, with (+) or without (−) RNase treatment. Each sample contains 1% triton X100 to disintegrate the NLPs and release replicon. In lane 6, the RNA migration may be affected by the highly charged cationic lipid, MVL5, which slowed RNA migration out of the well at a 500:1 ratio. DMPC with 5% MVL5 was made with ApoE4 scaffold protein. Cationic-NLPs comprised of DMPC with 10% DDAB, and DMPC with 20% DMTAP are made with ApoA1 scaffold protein. 0.1 μg RNA is loaded per lane. RNA alone is shown as a control.

FIG. 15 shows a graph of RT-PCR cycle threshold (Ct) results for exemplary NLPs formulated with RNA replicon at varying NLP:Replicon molar ratios. As a control, RNA alone (unformulated with NLP) was treated with RNase (gray bar) or without RNase (black bar). Each sample contains 5 ng of RNA. DMPC with 5% MVL5 was formulated with ApoE4 scaffold protein. DMPC with 10% DDAB, and DMPC with 20% DMTAP were formulated with ApoA1 scaffold protein.

FIG. 16 shows exemplary images of bioluminescence levels 4 to 5 days after mice were injected intramuscularly with cationic-NLP:Replicon expressing luciferase, or unformulated replicon RNA expressing luciferase (without cationic-NLP). Images are shown of mice that received either unformulated RNA replicon (1 μg or 30 μg), or cationic-NLP:Replicon comprising 80:20 DMPC:DMTAP, 80:20 DMPC:DMTAP plus 20% GMO, 80:20 DMPC:MVL5, 90:10 DMPC:DDAB, or 95:5 DMPC:MVL5. All cationic NLP formulations were injected with 1 μg RNA replicon. 1 μg RNA alone and 30 μg RNA alone are used as controls. The scaffold proteins ApoA1 or ApoE4 have been used as indicated on the figure.

FIG. 17 shows an exemplary graph of in vivo luciferase activity, assessed by bioluminescence (maximum total flux in photons per second, p/s) in groups of female BALB/c mice injected intramuscularly with either unformulated replicon RNA expressing luciferase (without cationic-NLP) at doses of 1 μg or 30 μg, or 1 μg RNA formulated with various cationic NLPs. A baseline control depicts mice that did not show any bioluminescent signal. The top cationic NLPs formulated with 1 μg RNA showed comparable bioluminescence to 30 μg naked RNA control. All NLP:Replicon formulations are at a 250:1 (NLP:RNA) molar ratio, except (A1, DMPC, DMTAP 20%, GMO 20%) which was formulated at a 50:1 (NLP:RNA) molar ratio. Each dot represents one mouse. P-values indicate significant differences between the groups indicated. A1 and E4 indicate ApoA1 and ApoE4 protein scaffolds, respectively. Naked RNA indicates RNA alone, unformulated with NLPs.

FIG. 18 shows exemplary graphs of in vivo luciferase intensity of RNA replicons formulated with cationic NLPs, cationic telo-NLPs, and RNA alone. Each data point represents maximal total flux from a single animal. Full data sets (left graph) contains all data points, even when no signal is detected (presumably from variability in administration). Removing null data points (right graph) illustrates effect of formulation if successfully administered. Bars indicated geometric mean with 95% confidence intervals.

FIG. 19 shows an exemplary graph of in vivo luciferase intensity of RNA replicons formulated with NLPs categorized in low, medium, and high efficacy classes. Each data point represents maximal total flux from a single animal. Bars indicated geometric mean.

FIGS. 20A-20C shows exemplary results of a SEC screen of NLP formation upon additional of increasing ratios of three different telodendrimers. FIG. 20A shows the result of a SEC screen of NLP formation with ApoAI, DMPC, DDAB (10%). FIG. 20B shows the result of a SEC screen of NLP formation with apoE4, DMPC, MVL5 (2%). FIG. 20C shows the result of a SEC screen of NLP formation with apoAI, DMPC, DMEPC (40%). These telodendrimers were tested with cationic NLPs binned into high efficacy (FIG. 20A), medium efficacy (FIG. 20B), or low efficacy (FIG. 20C), based on previous in vivo assessments of luciferase intensity. SEC traces (plotting absorbance intensity as a function of retention time) were used to identify those formulations that formed the most homogeneous species of NLPs, based on the presence of a single SEC peak. Formulations chosen for subsequent in vivo testing are indicated with check marks.

### DETAILED DESCRIPTION

Provided herein are nanolipoprotein particles and related compositions, methods and systems.

The term “nanolipoprotein particle” “nanodisc” “rHDL” or “NLP” as used herein indicates a supramolecular complex formed by a membrane forming lipid arranged in a lipid bilayer stabilized by a scaffold protein. The membrane forming lipids and scaffold protein are components of the NLP. In particular the membrane forming lipid component is part of a total lipid component, (herein also membrane lipid component or lipid component) of the NLP together with additional lipids such as functionalized lipids and/or lysolipids, that can further be included in the NLPs as will be understood by a skilled person upon reading of the present disclosure. The scaffold protein component is part of a protein component of the NLP together with additional proteins such as membrane proteins, target proteins and other proteins that can be further included as components of the NLPs as will be understood by a skilled person upon reading of the present disclosure. Additional components can be provided as part of the NLP herein described as will be understood by a skilled person. In particular, the membrane lipid bilayer can attach membrane proteins or other amphipathic compounds through interaction of respective hydrophobic regions with the membrane lipid bilayer. The membrane lipid bilayer can also attach proteins or other molecules through anchor compounds or functionalized lipids as will be understood by a skilled person upon reading of the disclosure. In a nanolipoprotein particle, the membrane lipid bilayer can be confined in a discoidal configuration by the scaffold protein. Predominately discoidal in shape, nanolipoprotein particles typically have diameters between 5 to 25 nm, share uniform heights between 3 to 6 nm and can be produced in yields ranging between 30 to 90%. Techniques suitable for detecting the size and dimension of the NLP comprise Transmission Electron Microscopy (TEM), Atomic Force Microscopy (AFM) Dynamic Light Scattering (DLS) and additional techniques identifiable by a skilled person.

In particular, in embodiments herein described, the nanolipoprotein particle can be formed by a lipid bilayer confined in a discoidal configuration by a scaffold protein. In this configuration, the lipid bilayer confined by the scaffold protein can be 4-7 nanometers in thickness, the nanolipoprotein particle can have an overall diameter of 6-30 nanometers, and the scaffold protein on the particle can have a thickness of 1-2 nanometers. In some embodiments, an entire NLP structure can be up to 600 kilodaltons in weight.

The particular membrane forming lipid, scaffold protein, the lipid to protein ratio, and the assembly parameters determine the size and homogeneity of nanolipoprotein particles as will be understood by a skilled person. In the nanolipoprotein particle the membrane forming lipids are typically arranged in a membrane lipid bilayer confined by the scaffold protein in a discoidal configuration as will be understood by a skilled person.

The term “membrane forming lipid” or “amphipathic lipid” as used herein indicates a lipid possessing both hydrophilic and hydrophobic moieties that in an aqueous environment assembles into a lipid bilayer structure that consists of two opposing layers of amphipathic molecules known as polar lipids. Each polar lipid has a hydrophilic moiety, i.e. a polar group such as, a derivatized phosphate or a saccharide group, and a hydrophobic moiety, i.e., a long hydrocarbon chain. Exemplary polar lipids include phospholipids, sphingolipids, glycolipids, ether lipids, sterols, alkylphosphocholines and the like. Amphipathic lipids include but are not limited to membrane lipids, i.e. amphipathic lipids that are constituents of a biological membrane, such as phospholipids like dimyristoylphosphatidylcholine (DMPC) or dioleoylphosphoethanolamine (DOPE) or dioleoylphosphatidylcholine (DOPC), or dipalmitoylphosphatidylcholine (DPPC). In a preferred embodiment, the lipid is dimyristoylphosphatidylcholine (DMPC).

The term “scaffold protein” as used herein indicates any amphipathic protein that is capable of self-assembly with amphipathic lipids in an aqueous environment, organizing the amphipathic lipids into a bilayer disc, and comprise apolipoproteins, lipophorins, derivatives thereof (such as truncated and tandemly arrayed sequences) and fragments thereof (e.g. peptides or fragments) which maintains the amphipathic nature and capability of self-assembly, such as apolipoprotein E4 (22 Kd fragment), lipophorin III, apolipoprotein A-1 and the like. In general, scaffold proteins have an alpha helical secondary structure in which a plurality of hydrophobic amino acids form a hydrophobic face and a plurality of hydrophilic amino acids form an opposing hydrophilic face. In some embodiments, rationally designed amphipathic peptides and synthetic apolipoproteins which maintain an amphipathic structure and capability of self-assembly can serve as a scaffold protein of the NLP.

The term “apolipoprotein” as used herein indicates an amphipathic protein that binds lipids to form lipoproteins. The term “amphipathic” pertains to a molecule containing both hydrophilic and hydrophobic properties. Exemplary amphipathic molecules comprise molecules having hydrophobic and hydrophilic regions/portions in its structure. Examples of biomolecules which are amphipathic include but not limited to phospholipids, cholesterol, glycolipids, fatty acids, bile acids, saponins, and additional lipids identifiable by a skilled person. A “lipoprotein” as used herein indicates a biomolecule assembly that contains both proteins and lipids. In particular, in lipoproteins, the protein component surrounds or solubilizes the lipid molecules enabling particle formation. Exemplary lipoproteins include the plasma lipoprotein particles classified under high-density (HDL) and low-density (LDL) lipoproteins, which enable fats and cholesterol to be carried in the blood stream, the transmembrane proteins of the mitochondrion and the chloroplast, and bacterial lipoproteins. In particular, the lipid components of lipoproteins are insoluble in water, but because of their amphipathic properties, apolipoproteins such as certain Apolipoproteins A and Apolipoproteins B and other amphipathic protein molecules can organize the lipids in a bilayer orientation with exposed hydrophilic moieties, creating the lipoprotein particle that is itself water-soluble, and can thus be carried through water-based circulation (e.g. blood, lymph in vivo or in vitro). Apolipoproteins known to provide the protein components of the lipoproteins can be divided into six classes and several sub-classes, based on the different structures and functions. Exemplary apolipoprotein known to be able to form lipoproteins comprise Apolipoproteins A (apo A-I, apo A-II, apo A-IV, and apo A-V), Apolipoproteins B (apo B48 and apo B100), Apolipoproteins C (apo C-I, apo C-II, apo C-III, and apo C-IV), Apolipoproteins D, Apolipoproteins E, and Apolipoproteins H. For example, apolipoproteins B can form low-density lipoprotein particles, and have mostly beta-sheet structure and associate with lipid droplets irreversibly, while Apolipoprotein A1 comprise alpha helices and can associate with lipid droplets reversibly forming high-density lipoprotein particles.

The term “protein” as used herein indicates a polypeptide with a particular secondary and tertiary structure that can interact with another molecule and in particular, with other biomolecules including other proteins, DNA, RNA, lipids, metabolites, hormones, chemokines, and/or small molecules. The term “polypeptide” as used herein indicates an organic linear, circular, or branched polymer composed of two or more amino acid monomers and/or analogs thereof. The term “polypeptide” includes amino acid polymers of any length including full-length proteins and peptides, as well as analogs and fragments thereof. A polypeptide of three or more amino acids is also called a protein oligomer, peptide, or oligopeptide. In particular, the terms “peptide” and “oligopeptide” usually indicate a polypeptide with less than 100 amino acid monomers. In particular, in a protein, the polypeptide provides the primary structure of the protein, wherein the term “primary structure” of a protein refers to the sequence of amino acids in the polypeptide chain covalently linked to form the polypeptide polymer. A protein “sequence” indicates the order of the amino acids that form the primary structure. Covalent bonds between amino acids within the primary structure can include peptide bonds or disulfide bonds, and additional bonds identifiable by a skilled person. Polypeptides in the sense of the present disclosure are usually composed of a linear chain of alpha-amino acid residues covalently linked by peptide bond or a synthetic covalent linkage. The two ends of the linear polypeptide chain encompassing the terminal residues and the adjacent segment are referred to as the carboxyl terminus (C-terminus) and the amino terminus (N-terminus) based on the nature of the free group on each extremity. Unless otherwise indicated, counting of residues in a polypeptide is performed from the N-terminal end (NH₂-group), which is the end where the amino group is not involved in a peptide bond to the C-terminal end (—COOH group) which is the end where a COOH group is not involved in a peptide bond. Proteins and polypeptides can be identified by x-ray crystallography, direct sequencing, immunoprecipitation, and a variety of other methods as understood by a person skilled in the art. Proteins can be provided in vitro or in vivo by several methods identifiable by a skilled person. In some instances where the proteins are synthetic proteins in at least a portion of the polymer two or more amino acid monomers and/or analogs thereof are joined through chemically-mediated condensation of an organic acid (—COOH) and an amine (—NH₂) to form an amide bond or a “peptide” bond.

As used herein the term “amino acid”, “amino acid monomer”, or “amino acid residue” refers to organic compounds composed of amine and carboxylic acid functional groups, along with a side-chain specific to each amino acid. In particular, alpha- or α-amino acid refers to organic compounds composed of amine (—NH2) and carboxylic acid (—COOH), and a side-chain specific to each amino acid connected to an alpha carbon. Different amino acids have different side chains and have distinctive characteristics, such as charge, polarity, aromaticity, reduction potential, hydrophobicity, and pKa. Amino acids can be covalently linked to form a polymer through peptide bonds by reactions between the amine group of a first amino acid and the carboxylic acid group of a second amino acid. Amino acid in the sense of the disclosure refers to any of the twenty naturally occurring amino acids, non-natural amino acids, and includes both D an L optical isomers.

In embodiments herein described, cationic nanolipoprotein particles comprise one or more cationic lipids within the lipid bilayer also comprising one or more membrane forming lipids.

The term “cationic lipids” as used herein refers to a subset of polar lipids that are characterized as having a hydrophobic region to anchor in the bilayer and a polar or positively-charged head group that is presented on the bilayer surface. In some cases, the head groups are ionizable head groups that are uncharged at a pH above their pK but capable of becoming positively charged at a pH below their pK.

In some embodiments, cationic lipids herein described comprise lipids of Formula (I):

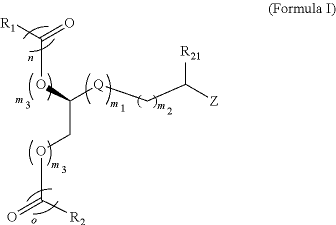

R1 and R2 are independently selected from H and a C₇-C₂₉ branched or straight, substituted or unsubstituted aliphatic carbon chain;
R₂₁ is H, OH, or a carboxy group;
Q in Formula I is selected from:

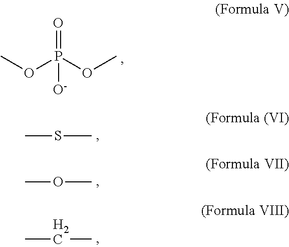

m₁=0-1; m₂=0-3; m₃=0-1 and
n and o are independently 0 and 1;
Z is a moiety of Formula (II) or Formula (III) or Formula (IV), wherein
the moiety of Formula (II) is

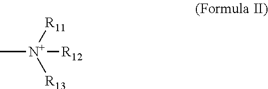

in which R₁₁, R₁₂ and R₁₃ are independently H or a C₁-C₄ branched or straight aliphatic carbon chain;
the moiety of Formula (III) is

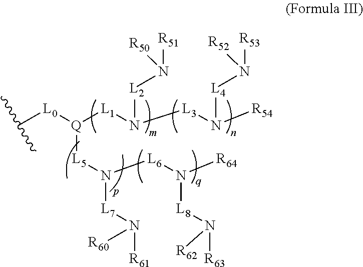

### Wherein

Q in Formula III is N or CH;
m, n, p and q are independently 0-3 and m+n+p+q is equal or greater than 1;
L₀-L₈ are independently a divalent group selected from C₁₋₆ alkylidenyl, C₁₋₆ heteroalkylidenyl, C₃₋₆ cycloalkylidenyl, and C₃₋₆ heterocycloalkylidenyl, wherein each of the alkylidenyl, heteroalkylidenyl, cycloalkylidenyl, and heterocycloalkylidenyl is optionally substituted with one or more substituents independently selected from C₁₋₄ alkyl, haloalkyl, C₁₋₄ alkoxy, amino, alkylamino, dialkylamino, oxo groups;
R₅₀-R₅₄, R₆₀-R₆₄ are independently a monovalent group selected from H, C₁₋₆ alkyl, C₁₋₆ heteroalkyl, C₃₋₆ cycloalkyl, and C₃₋₆ heterocycloalkyl, wherein each of the alkyl, heteroalkyl, cycloalkyl, and heterocycloalkyl is optionally substituted with one or more substituents independently selected from C₁₋₄ alkyl, haloalkyl, C₁₋₄ alkoxy, amino, C₁₋₄ alkylamino, C₁₋₄ dialkylamino, or a oxo group; and
the moiety of Formula (IV) is

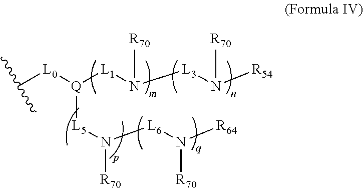

in which Q in Formula IV is N or CH;
m, n, p and q are independently 0-3 and m+n+p+q is equal or greater than 1;
L₀-L₁; L₃ and L₅-L₆ are independently a divalent group selected from C₁₋₆ alkylidenyl, C₁₋₆ heteroalkylidenyl, C₃₋₆ cycloalkylidenyl, and C₃₋₆ heterocycloalkylidenyl, wherein each of the alkylidenyl, heteroalkylidenyl, cycloalkylidenyl, and heterocycloalkylidenyl is optionally substituted with one or more substituents independently selected from C₁₋₄ alkyl, haloalkyl, C₁₋₄ alkoxy, amino, alkylamino, dialkylamino, oxo groups;
R₅₄, R₆₄ and R₇₀ are independently a monovalent group selected from H, C₁₋₆ alkyl, C₁₋₆ heteroalkyl, C₃₋₆ cycloalkyl, and C₃₋₆ heterocycloalkyl, wherein each of the alkyl, heteroalkyl, cycloalkyl, and heterocycloalkyl is optionally substituted with one or more substituents independently selected from C₁₋₄ alkyl, haloalkyl, C₁₋₄ alkoxy, amino, C₁₋₄ alkylamino, C₁₋₄ dialkylamino, or a oxo group.

As used herein, the term “aliphatic” refers to an alkyl, alkenyl or alkynyl group which can be substituted or unsubstituted, linear, branched or cyclic.

As used herein the term “alkyl” as used herein refers to a linear, branched, or cyclic, saturated hydrocarbon group formed by a carbon chain. As used herein the term “carbon chain” indicates a linear or branched line of connected carbon atoms. An alkyl carbon chain typically although not necessarily containing 1 to about 18 carbon atoms (±1 atom), preferably 1 to about 6 carbon atoms (±1 atom), such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 6 carbon atoms (±1 atom). The term “cycloalkyl” intends a cyclic alkyl group, typically having 4 to 8, preferably 5 to 7, carbon atoms. The term “substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl and lower alkyl, respectively.

As used herein the term “alkenyl” indicates a linear, branched, or cyclic hydrocarbon group that contains at least one carbon-carbon double bond. As used herein the term “alkynyl” indicates a linear, branched, or cyclic hydrocarbon group that contains at least one carbon-carbon triple bond.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 12 carbon atoms, and particularly preferred aryl groups contain 5 to 6 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituents in which at least one carbon atom is replaced with a heteroatom, such as nitrogen, oxygen or sulfur.

As used herein the terms “heteroatom-containing” or “hetero-” indicated in connection with a group, refers to a hydrocarbon group in which one or more carbon atoms is replaced with an atom other than carbon, e.g., nitrogen, oxygen, sulfur, phosphorus or silicon, typically nitrogen, oxygen or sulfur. Exemplary “heteroatoms” comprise as N, O, S and P, and can be present in a compound by a covalent bond to each of two carbon atoms, thus interrupting the two carbon atoms. Accordingly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. It should be noted that a “heterocyclic” group or compound may or may not be aromatic, and further that “heterocycles” may be monocyclic, bicyclic, or polycyclic as described above with respect to the term “aryl.” Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, and addition group identifiable by a skilled person.

The term “aralkyl” as used herein refers to an alkyl group with an aryl substituent, and the term “alkaryl” as used herein refers to an aryl group with an alkyl substituent, wherein “aryl” and “alkyl” are as defined above. In some embodiments, alkaryl and aralkyl groups contain 6 to 12 carbon atoms, and particularly alkaryl and aralkyl groups contain 6 to 16 carbon atoms. Alkaryl groups include, for example, p-methylphenyl, 2,4-dimethylphenyl, p-cyclohexylphenyl, 3-ethyl-cyclopenta-1,4-diene, and the like. Examples of aralkyl groups include, without limitation, benzyl, 2-phenyl-ethyl, 3-phenyl-propyl, 4-phenyl-butyl, 5-phenyl-pentyl, 4-phenylcyclohexyl, and the like. The terms “alkaryloxy” and “aralkyloxy” refer to substituents of the formula —OR wherein R is alkaryl or aralkyl, respectively, as defined.

The terms “cyclic” and “ring” refer to alicyclic or aromatic groups that may or may not be substituted and/or heteroatom containing, and that may be monocyclic, bicyclic, or polycyclic. The term “alicyclic” is used in the conventional sense to refer to an aliphatic cyclic moiety, as opposed to an aromatic cyclic moiety, and may be monocyclic, bicyclic or polycyclic.

Unless otherwise indicated, the term “substituted” as in “substituted alkyl,” “substituted aryl,” and the like, is meant that in the, alkyl, aryl, or other moiety, at least one hydrogen atom bound to a carbon (or other) atom is replaced with one or more non-hydrogen substituents. As used herein, a “substituent” is an atom or group of atoms substituted in place of a hydrogen atom on the main chain of a hydrocarbon. Examples of such substituents include, without limitation: functional groups such as, hydroxyl, sulfhydryl, C₁-C₁₂ alkoxy, C₂-C₁₂ alkenyloxy, C₂-C₁₂ alkynyloxy, C₅-C₁₂ aryloxy, C₆-C₁₂ aralkyloxy, C₆-C₁₂ alkaryloxy, acyl (including C₂-C₁₂ alkylcarbonyl (—CO-alkyl) and C₆-C₁₂ arylcarbonyl (—CO-aryl)), acyloxy (—O-acyl, including C₂-C₁₂ alkylcarbonyloxy (—O—CO— alkyl) and C₆-C₁₂ arylcarbonyloxy (—O—CO-aryl)), C₂-C₁₂ alkoxycarbonyl (—(CO)—O-alkyl), C₆-C₁₂ aryloxycarbonyl (—(CO)—O-aryl), C₂-C₁₂ alkylcarbonato (—O—(CO)—O-alkyl), C₆-C₁₂ arylcarbonato (—O—(CO)—O-aryl), carboxy (—COOH), carboxylato (—COO⁻), carbamoyl (—(CO)—NH₂), mono-(C₁-C₁₂ alkyl)-substituted carbamoyl (—(CO)—NH(C₁-C₁₂ alkyl)), di-(C₁-C₁₂ alkyl)-substituted carbamoyl (—(CO)—N(C₁-C₁₂ alkyl)₂), mono-(C₅-C₁₂ aryl)-substituted carbamoyl (—(CO)—NH-aryl), di-(C₅-C₁₂ aryl)-substituted carbamoyl (—(CO)—N(C₅-C₁₂ aryl)₂), di-N—(C₁-C₆ alkyl), N—(C₅-C₁₂ aryl)-substituted carbamoyl, thiocarbamoyl (—(CS)—NH₂), mono-(C₁-C₁₂ alkyl)-substituted thiocarbamoyl (—(CO)—NH(C₁-C₁₂ alkyl)), di-(C₁-C₁₂ alkyl)-substituted thiocarbamoyl (—(CO)—N(C₁-C₆ alkyl)₂), mono-(C₅-C₁₂ aryl)-substituted thiocarbamoyl (—(CO)—NH-aryl), di-(C₅-C₆ aryl)-substituted thiocarbamoyl (—(CO)—N(C₅-C₆ aryl)₂), di-N—(C₁-C₆ alkyl), N—(C₅-C₆ aryl)-substituted thiocarbamoyl, carbamido (—NH—(CO)—NH₂), cyano (—C≡N), cyanato (—O—C≡N), thiocyanato (—S—C≡N), formyl (—(CO)—H), thioformyl (—(CS)—H), amino (—NH₂), mono-(C₁-C₁₂ alkyl)-substituted amino, di-(C₁-C₁₂ alkyl)-substituted amino, mono-(C₅-C₁₂ aryl)-substituted amino, di-(C₅-C₆ aryl)-substituted amino, C₂-C₁₂ alkylamido (—NH—(CO)-alkyl), C₆-C₁₂ arylamido (—NH—(CO)-aryl), imino (—CR═NH where R=hydrogen, C₁-C₁₂ alkyl, C₅-C₁₂ aryl, C₆-C₁₂ alkaryl, C₆-C₁₂ aralkyl, etc.), C₂-C₁₂ alkylimino (—CR═N(alkyl), where R=hydrogen, C₁-C₁₂ alkyl, C₅-C₁₂ aryl, C₆-C₁₂ alkaryl, C₆-C₂ aralkyl, etc.), arylimino (—CR═N(aryl), where R=hydrogen, C₁-C₁₂ alkyl, C₅-C₁₂ aryl, C₆-C₁₂ alkaryl, C₆-C₁₂ aralkyl, etc.), nitro (—NO₂), nitroso (—NO), sulfo (—SO₂—OH), sulfonato (—SO₂—O—), C₁-C₁₂ alkylsulfanyl (—S-alkyl; also termed “alkylthio”), C₅-C₁₂ arylsulfanyl (—S-aryl; also termed “arylthio”), C₁-C₁₂ alkylsulfinyl (—(SO)-alkyl), C₅-C₁₂ arylsulfinyl (—(SO)-aryl), C₁-C₁₂ alkylsulfonyl (—SO₂-alkyl), C₅-C₁₂ arylsulfonyl (—SO₂-aryl), boryl (—BH₂), borono (—B(OH)₂), boronato (—B(OR)₂ where R is alkyl or other hydrocarbyl), phosphono (—P(O)(OH)₂), phosphonato (—P(O)(O)₂), phosphinato (—P(O)(O)), phospho (—PO₂), phosphino (—PH₂), silyl (—SiR₃ wherein R is hydrogen or hydrocarbyl), and silyloxy (—O-silyl); and the hydrocarbyl moieties C₁-C₁₂ alkyl (preferably C₁-C₁₂ alkyl, more preferably C₁-C₆ alkyl), C₂-C₁₂ alkenyl (preferably C₂-C₁₂ alkenyl, more preferably C₂-C₆ alkenyl), C₂-C₁₂ alkynyl (preferably C₂-C₁₂ alkynyl, more preferably C₂-C₆ alkynyl), C₅-C₁₂ aryl (preferably C₅-C₁₂ aryl), C₆-C₁₂ alkaryl (preferably C₆-C₁₂ alkaryl), and C₆-C₁₂ aralkyl (preferably C₆-C₁₂ aralkyl).

In some embodiments, the cationic lipids comprise lipids of Formula (IX):

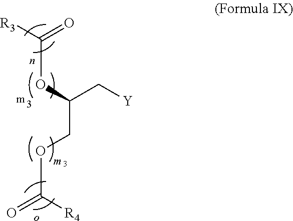

wherein
R₃, R₄ are independently a C₇-C₂₉ branched or straight, substituted or unsubstituted aliphatic carbon chain;
Y is a moiety of formula (III), (IV) or (X), wherein the moiety of Formula (X) is

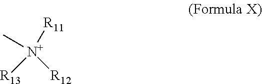

in which R₁₁, R₁₂ and R₁₃ are independently H or a C₁-C₄ branched or straight aliphatic carbon chain;
n and o are independently 0 and 1; and m₃=0 or 1.

In some embodiments, cationic lipids comprise lipids of Formula (XI)

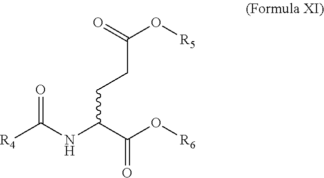

wherein R₄, R₅ and R₆ are independently H and C₇-C₂₉ branched or straight, substituted or unsubstituted aliphatic carbon chain, and at least one of R₄, R₅ and R₆ contains at least one amino nitrogen and wherein at least one of R₄, R₅ and R₆ is H.

In some embodiments, cationic lipids comprise lipids of Formula (XII)

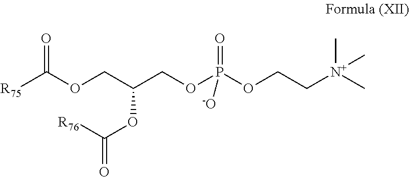

wherein R₇₅ and R₇₆ are independently selected from a C₇ to C₁₃ alkyl, C₇ to C₁₃ alkenyl group.

In some embodiments, cationic lipids comprise lipids of Formula (XIII)

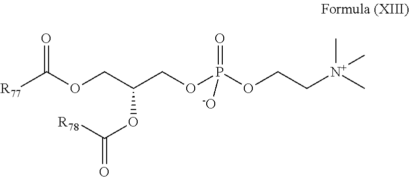

wherein R₇₇ and R₇₈ are independently selected from a C₁₄ to C₁₉ alkyl, C₁₄ to C₁₉ alkenyl group.

In some embodiments, cationic lipids herein described comprise one or more multivalent cationic lipids. The term “multivalent cationic lipid” refers to a subset of cationic lipid having a hydrophobic region and a head group bearing a positive charge varied from +2 to +16. In contrast, the term “monovalent cationic lipid” therefore refers to a subset of cationic lipid having a hydrophobic region and a head group bearing a positive charge of +1.

In some embodiments, the multivalent cationic lipids herein described have a head group of Formula (XIV):

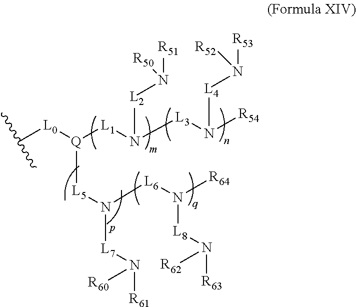

wherein
Q in Formula XIV is N or CH;
m, n, p and q are independently 0-3 and m+n+p+q is equal or greater than 1;
L₀-L₈ are independently a divalent group selected from C₁₋₆ alkylidenyl, C₁₋₆ heteroalkylidenyl, C₃₋₆ cycloalkylidenyl, and C₃₋₆ heterocycloalkylidenyl, wherein each of the alkylidenyl, heteroalkylidenyl, cycloalkylidenyl, and heterocycloalkylidenyl is optionally substituted with one or more substituents independently selected from C₁₋₄ alkyl, haloalkyl, C₁₋₄ alkoxy, amino, alkylamino, dialkylamino, oxo groups;
R₅₀-R₅₄, R₆₀-R₆₄ are independently a monovalent group selected from H, C₁₋₆ alkyl, C₁₋₆ heteroalkyl, C₃₋₆ cycloalkyl, and C₃₋₆ heterocycloalkyl, wherein each of the alkyl, heteroalkyl, cycloalkyl, and heterocycloalkyl is optionally substituted with one or more substituents independently selected from C₁₋₄ alkyl, haloalkyl, C₁₋₄ alkoxy, amino, C₁₋₄ alkylamino, C₁₋₄ dialkylamino, or a oxo group.

In some embodiments, the head group of the multivalent cationic lipids herein described can have a formula (XV):

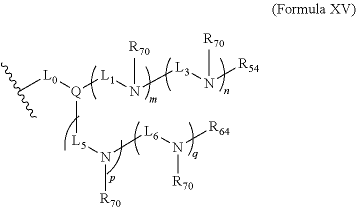

Q in Formula XV is N or CH;
m, n, p and q are independently 0-3 and m+n+p+q is equal or greater than 1;
L₀-L₈ are independently a divalent group selected from C₁₋₆ alkylidenyl, C₁₋₆ heteroalkylidenyl, C₃₋₆ cycloalkylidenyl, and C₃₋₆ heterocycloalkylidenyl, wherein each of the alkylidenyl, heteroalkylidenyl, cycloalkylidenyl, and heterocycloalkylidenyl is optionally substituted with one or more substituents independently selected from C₁₋₄ alkyl, haloalkyl, C₁₋₄ alkoxy, amino, alkylamino, dialkylamino, oxo groups;
R₅₄, R₆₄ and R₇₀ are independently a monovalent group selected from H, C₁₋₆ alkyl, C₁₋₆ heteroalkyl, C₃₋₆ cycloalkyl, and C₃₋₆ heterocycloalkyl, wherein each of the alkyl, heteroalkyl, cycloalkyl, and heterocycloalkyl is optionally substituted with one or more substituents independently selected from C₁₋₄ alkyl, haloalkyl, C₁₋₄ alkoxy, amino, C₁₋₄ alkylamino, C₁₋₄ dialkylamino, or a oxo group.

In some embodiments, the multivalent cationic lipids herein described comprise lipids of formula (XVI):

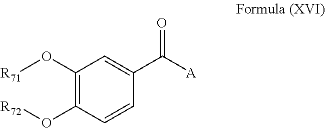

wherein R₇₁ and R₇₂ are independently C₆ to ₁₈ alkyl or alkenyl group and A is a moiety of Formula (III) or Formula (IV).

In some particular embodiments, the multivalent cationic lipids herein described comprise lipids of MVL5 of formula (XVII):

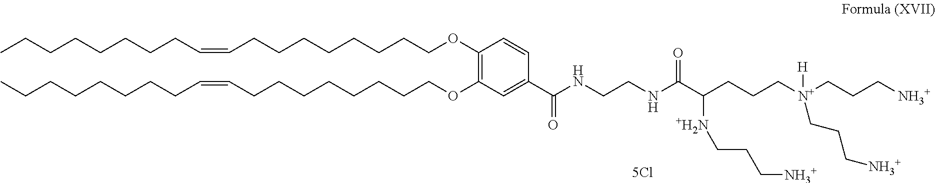 [SMILES: CCCCCCCC/C=C\CCCCCCCCOc1ccc(C(=O)NCCNC(=O)C(CCC[NH+](CCC[NH3+])CCC[NH3+])[NH2+]CCC[NH3+])cc1OCCCCCCCC/C=C\CCCCCCCC]

In some embodiments herein described, the multivalent cationic lipids can have a highly charged dendritic head group wherein the dendritic head group comprises repetitive branch units each branch unit bearing at least one positively charged group. For example, the multivalent cationic lipids can comprise lipids of MVLBG2.

In some embodiments, the multivalent cationic lipids herein described comprise lipids of Formula (XVIII)

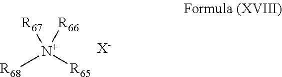

wherein
R₆₅ and R₆₆ are independently C₁ to ₄ alkyl or alkenyl group;
R₆₇ and R₆₈ are independently C₈ to ₁₉ alkyl or alkenyl group; and
X is an ion including F⁻, Cl⁻, Br⁻, NO₃⁻, and ClO₄⁻.

In embodiments herein described, cationic NLPs comprise a scaffold protein and a lipid component comprising cationic lipids and membrane forming lipids in ratios and proportions that would be identifiable by a skilled person upon reading of the present disclosure.

In some embodiments, cationic NLPs herein described have a lipid component to scaffold molar ratio ranging from 20:1 to 240:1, preferably from 40:1 to 240:1, depending on the scaffold protein and the lipid component used as will be understood by a skilled person. Within the lipid component of the NLPs, the cationic lipids are comprised in about 1%-60% (±0.5, preferably from 5% to 40% (±0.5%, of the total lipids in the lipid component including the cationic lipids and the membrane forming lipids.

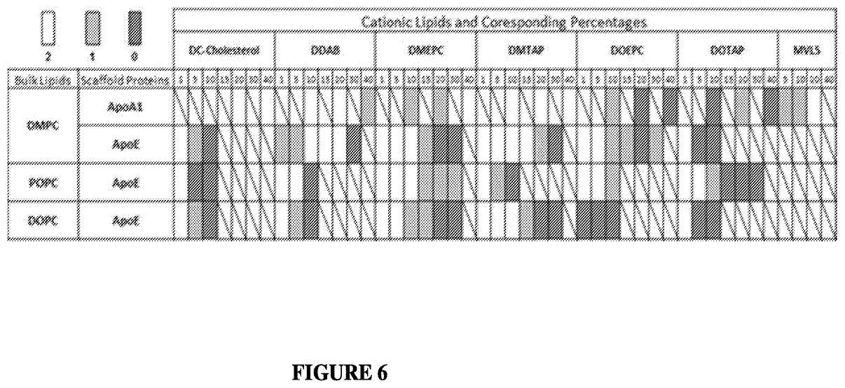

In some particular embodiments, the cationic NLPs herein described have apoE422k or apoA1 as scaffold protein and DMPC, POPC, DOPC as the membrane forming lipid. In cases when DMPC is used as the membrane forming lipids, cationic lipids such as DC-cholesterol, DDAB, DMEPC, DMTAP, DOEPC, DOTAP and MVL5 are in a percentage from 1% up to 40% of the lipid component (see FIG. 6 and Example 2), preferably with DMEPC, DDAB and DMTAP at a percentage from 1% to 20%. In cases when POPC or DOPC is used as the membrane forming lipids, cationic lipids such as DC-cholesterol, DDAB, DMEPC, DMTAP, DOEPC, DOTAP and MVL5 are in a percentage from 1% up to 30% of the lipid component.

In some embodiments, cationic NLPs herein described have a lipid component comprising membrane forming lipids in an amount from 99 to 40 mol % of the lipid component and the cationic lipids in an amount from 1 to 60 mol % of the lipid component. In some particular embodiments, the lipid component comprises membrane forming lipids in an amount from 95 to 60 mol % of the lipid component and the cationic lipids in an amount from 5 to 40 mol % of the lipid component.

In preferred embodiments, the NPLs herein described can have a membrane forming lipid:cationic lipid:scaffold protein ratio range between 19:1:1 and 96:144:1, preferably 136:34:1, with exact molar ratios depending on the optimal lipid:protein ratio for that lipid mixture and scaffold protein identifiable by a skilled person upon reading of the present disclosure.

In some embodiments, the cationic lipids are comprised within an NLP herein described in a molar lipid concentration about 1-60% (±0.5%, preferably about 5-20% (±0.5%. In some embodiments, the cationic lipids can be comprised within an NLP herein described in an amount of 20% of the total lipid content of the NLP and the membrane forming lipids can be comprised within an NLP herein described in an amount of 80% of the total lipid content.

In some embodiments, cationic-NLP herein described can be provided with a concentration of monovalent and/or multivalent cationic lipid selected based on valency of the cationic lipid so that the greater the multivalency, the lower the overall lipid % in the NLP. In particular, in some embodiments, the cationic-NLPs can comprise cationic lipids in concentrations of 1% to 5% such as 1%, 2%, or 5% of multivalent cationic lipids (such as MVL5) with 99, 98, or 95% of membrane forming lipids (such as DMPC). In some embodiments, the cationic-NLPs can comprise cationic lipids in concentrations of at least 5% such as 5%, 10%, and 20% of monovalent cationic lipids (such as DDAB) with 95, 90, or 80% of membrane forming lipids (such as DMPC).

In some embodiments, the membrane forming lipids component of the lipid component lipids such as phospholipids, preferably including at least one phospholipid, typically soy phosphatidylcholine, egg phosphatidylcholine, soy phosphatidylglycerol, egg phosphatidylglycerol, palmitoyl-oleoyl-phosphatidylcholine distearoylphosphatidylcholine, or distearoylphosphatidylglycerol. Other useful phospholipids include, e.g., phosphatidylcholine, phosphatidylglycerol, sphingomyelin, phosphatidylserine, phosphatidic acid, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, stearoyl-palmitoyl-phosphatidylcholine, di-palmitoyl-phosphatidylethanolamine, distearoyl-phosphatidylethanolamine, di-myrstoyl-phosphatidylserine, and dioleyl-phosphatidylcholine.

Additionally exemplary membrane forming lipids that can be comprised in various combinations together with one or more lysolipids comprise 1,2-dipalmitoyl-sn-glycero-3-phosphocholine, 1,2-dimyristoyl-sn-glycero-3-phosphocholine, 1,2-dilauroyl-sn-glycero-3-phosphocholine, 1,2-didecanoyl-sn-glycero-3-phosphocholine, 1,2-dierucoyl-sn-glycero-3-phosphocholine, 1,2-dioleoyl-sn-glycero-3-phosphocholine, 1,2-dipalmitoleoyl-sn-glycero-3-phosphocholine, 1,2-dimyristoleoyl-sn-glycero-3-phosphocholine, 1-stearoyl-2-oleoyl-sn-glycero-3-phosphocholine, 1-stearoyl-2-myristoyl-sn-glycero-3-phosphocholine, 1-palmitoyl-2-oleoyl-sn-glycero-3-phosphocholine, egg phosphatidylcholine extracts, soy phosphatidylcholine extracts, heart phosphatidylcholine extracts, brain phosphatidylcholine extracts, liver phosphatidylcholine extracts, 1,2-distearoyl-sn-glycero-3-phosphate, 1,2-dipalmitoyl-sn-glycero-3-phosphate, 1,2-dimyristoyl-sn-glycero-3-phosphate, 1,2-dilauroyl-sn-glycero-3-phosphate, 1-palmitoyl-2-oleoyl-sn-glycero-3-phosphate, 1-stearoyl-2-oleoyl-sn-glycero-3-phosphate, 1,2-dipalmitoyl-sn-glycero-3-phosphoethanolamine, 1,2-dimyristoyl-sn-glycero-3-phosphoethanolamine, 1,2-dilauroyl-sn-glycero-3-phosphoethanolamine, 1,2-dioleoyl-sn-glycero-3-phosphoethanolamine, 1,2-dipalmitoleoyl-sn-glycero-3-phosphoethanolamine, Egg phosphatidylethanolamine extract, soy phosphatidylethanolamine extract, heart phosphatidylethanolamine extract, brain phosphatidylethanolamine extract, 1,2-distearoyl-sn-glycero-3-phospho-(1′-rac-glycerol), 1,2-dioleoyl-sn-glycero-3-phospho-(1′-rac-glycerol), 1,2-dipalmitoyl-sn-glycero-3-phospho-(1′-rac-glycerol), 1,2-dimyristoyl-sn-glycero-3-phospho-(1′-rac-glycerol), 1,2-dilauroyl-sn-glycero-3-phospho-(1′-rac-glycerol), 1-palmitoyl-2-oleoyl-sn-glycero-3-phospho-(1′-rac-glycerol), egg phosphatidylglycerol extract, soy phosphatidylglycerol extract, 1,2-distearoyl-sn-glycero-3-phospho-L-serine, 1,2-dioleoyl-sn-glycero-3-phospho-L-serine, 1,2-dipalmitoyl-sn-glycero-3-phospho-L-serine, 1,2-dimyristoyl-sn-glycero-3-phospho-L-serine, 1,2-dilauroyl-sn-glycero-3-phospho-L-serine, 1-palmitoyl-2-oleoyl-sn-glycero-3-phospho-L-serine, soy phosphatidylserine extract, brain phosphatidylserine extract, 2-((2,3-bis(oleoyloxy)propyl)dimethylammonio)ethyl hydrogen phosphate, cholesterol, ergosterol, sphingolipids, ceramides, sphingomyelin, gangliosides, glycosphingolipids, 1,2-dioleoyl-3-trimethylammonium-propane, 1,2-di-O-octadecenyl-3-trimethylammonium propane.

In some embodiments, non-phosphorus containing lipids can also be used as membrane forming lipids in the NLPs herein described, e.g. stearylamine, docecylamine, acetyl palmitate, and fatty acid amides. Additional membrane forming lipids suitable for use in providing NLPs are well known to persons of ordinary skill in the art and are cited in a variety of well-known sources, e.g., McCutcheon's Detergents and Emulsifiers and McCutcheon's Functional Materials, Allured Publishing Co., Ridgewood, N.J., both of which are incorporated herein by reference.

In some embodiments, the cationic lipid component comprises lipids such as DC-cholesterol (3β-[N—(N′,N′-dimethylaminoethane)-carbamoyl]cholesterol hydrochloride), DDAB (dimethyldioctadecylammonium (Bromide Salt)), DMEPC (1,2-dimyristoleoyl-sn-glycero-3-ethylphosphocholine), DOEPC (1,2-dioleoyl-sn-glycero-3-ethylphosphocholine) (the EPC family further includes 16:0-18:1, 18:0, 16:0, 14:0, 12:0 variants), DMTAP (1,2-dimyristoyl-3-trimethylammonium-propane), DOTAP (1,2-dioleoyl-3-trimethylammonium-propane) (the TAP family further includes 18:0, 16:0, 14:0 variants), MVL5 (N1-[2-((1S)-1-[(3-aminopropyl)amino]-4-[di(3-amino-propyl)amino]butylcarboxamido)ethyl]-3,4-di[oleyloxy]-benzamide), DODAP (1,2-dioleoyl-3-dimethylammonium-propane), DMDAP (1,2-dimyristoyl-3-dimethylammonium-propane), DPDAP (1,2-dipalmitoyl-3-dimethylammonium-propane), DSDAP (1,2-distearoyl-3-dimethylammonium-propane), DOTMA (1,2-di-O-octadecenyl-3-trimethylammonium propane), and others identifiable by a person of ordinary skill in the art. Additional cationic lipids are commercially available or identifiable by a skilled person upon reading of the present disclosure (Semple C. S. et. al., Nature Biotechnology, 2010, Vol. 28:172-178).

In some embodiments various combinations and ratios of membrane forming lipids and cationic lipids can be comprised within a cationic NLP herein described, such as DMPC and DMTAP at cationic lipid ratio range of 1 to 40, DMPC and DOTAP at cationic lipid ratio range of 1 to 20, DMPC and DC-cholesterol at cationic lipid ratio range of 1 to 40, DMPC and DDAB at cationic lipid ratio range of 1 to 20, DMPC and DMEPC at cationic lipid ratio range of 1 to 40, DMPC and DODAP at cationic lipid ratio range of 1 to 40, DMPC and DOEPC at cationic lipid ratio range of 1 to 30, DMPC and DOTMA at cationic lipid ratio range of 1 to 20, DMPC and MVL5 at cationic lipid ratio range of 0.2 to 20. In some embodiments, the total amount of cationic lipids is less than 60% of the total amount of lipids used in the NLPs.

The above ratio between membrane forming lipid DMPC and various cationic can be derived for other membrane forming lipids such as DOPC, POPC, DOPE, DPPC and natural lipids extracts such as SoyPC and EggPC and a mixture thereof as will be understood for a skilled person, and cationic lipid ratios can range from 0.2 to 60 with a total cationic lipid which in some instances can amount to less than 60% of the total lipid of the cationic-NLPs herein described

In some embodiments, the scaffold proteins can contain amino acid additions, deletions, or substitutions. In other embodiments, the scaffold proteins can be derived from various species and more particularly derived from human, mouse, rat, guinea pig, rabbit, cow, horse, pig, dog, and non-human primates.

In some embodiments various combinations of membrane forming lipids and cationic lipids in accordance with the disclosure can be comprised within an NLP stabilized by scaffold proteins such as human derived apoE4, truncated versions of human derived apoE4 (e.g. apoE422k), human derived apoE3, truncated versions of human derived apoE3 (e.g. apoE322k), human derived apoE2, truncated versions of human derived apoE2 (e.g. apoE222k), human derived apoA1, truncated versions of human derived apoA1 (e.g. Δ49ApoA1, MSP1, MSP1T2, MSP1E3D1), mouse derived apoE4, truncated versions of mouse derived apoE4 (e.g. apoE422k), mouse derived apoE3, truncated versions of mouse derived apoE3 (e.g. apoE322k), mouse derived apoE2, truncated versions of mouse derived apoE2 (e.g. apoE222k), mouse derived apoA1, truncated versions of mouse derived apoA1 (e.g. Δ49ApoA1, MSP1, MSP1T2, MSP1E3D1), rat derived apoE4, truncated versions of rat derived apoE4 (e.g. apoE422k), rat derived apoE3, truncated versions of rat derived apoE3 (e.g. apoE322k), rat derived apoE2, truncated versions of rat derived apoE2 (e.g. apoE222k), rat derived apoA1, truncated versions of rat derived apoA1 (e.g. Δ49ApoA1, MSP1, MSP1T2, MSP1E3D1), lipophorins (e.g. B. mori, M. sexta), synthetic linear or cyclic peptides that mimic the function of apolipoproteins. Other apolipoproteins, as will be understood for a skilled person, can be used to form NLP, including but not limited to apoB and apoC.

In some embodiments, the scaffold protein is formed by amphipathic peptides and/or synthetic apolipoproteins which are configured to maintain an amphipathic structure and capability of self-assembly. In particular, in those embodiments, the peptides and/or synthetic apolipoprotein are configured and selected to provide a plurality of helical segments each having a primary structure configured to form an alpha helix secondary structure. In the alpha helix secondary structure of at least one helical segment, the peptides and/or synthetic apolipoprotein comprise a plurality of hydrophobic amino acids and a plurality of hydrophilic amino acids positioned in the primary structure to provide an amphipathic alpha helix secondary structure, with the plurality of hydrophobic amino acids forming an hydrophobic amino acid cluster and the plurality hydrophilic amino acids forming an hydrophilic amino acid cluster. In some of those embodiments, the scaffold proteins can be peptides derived from apolipoproteins, and can contain amino acid additions, deletions, or substitutions. In other embodiments, these peptides have no sequence homology to apolipoproteins but can be structural analogs. In some embodiments, the peptides can be prepared with L- or D-amino acids. In embodiments where the scaffold protein comprises one or more peptides the skilled person would be able to identify the ratios of peptides based on the length and number of peptides and apolipoproteins and on a desired dimension of the nanolipoprotein particles upon reading of the present disclosure. Additional description of scaffold proteins can be found in PCT/US2015/051172 published on Mar. 16, 2017 as WO2017/044899 incorporated herein by reference in its entirety.

In some embodiments various combinations of membrane forming lipids and cationic lipids in accordance with the disclosure can be comprised within an NLP stabilized by different scaffold proteins, DMPC and DMTAP (ratio range of 1 to 40×) with human derived apoE422k (lipid:scaffold protein range 40:1 to 200:1), DMPC and DOTAP (ratio range of 1× to 20×) with human derived apoE422k (lipid:scaffold protein range 40:1 to 200:1), DMPC and DC-cholesterol (ratio range of 1× to 40×) with human derived apoE422k (lipid:scaffold protein range 40:1 to 200:1), DMPC and DDAB (ratio range of 1× to 20×) with human derived apoE422k (lipid:scaffold protein range 40:1 to 200:1), DMPC and DMEPC (ratio range of 1× to 40×) with human derived apoE422k (lipid:scaffold protein range 40:1 to 200:1), DMPC and DODAP (ratio range of 1× to 40×) with human derived apoE422k (lipid:scaffold protein range 40:1 to 200:1), DMPC and DOEPC (ratio range of 1× to 30×) with human derived apoE422k (lipid:scaffold protein range 40:1 to 200:1), DMPC and DOPE (ratio range of 1× to 40×) with human derived apoE422k (lipid:scaffold protein range 40:1 to 200:1), DMPC and DOTMA (ratio range of 1× to 20×) with human derived apoE422k (lipid:scaffold protein range 40:1 to 200:1), DMPC and MVL5 (ratio range of 0.2× to 20×) with human derived apoE422k (lipid:scaffold protein range 40:1 to 200:1).

The above ratios between membrane forming lipid DMPC, various cationic lipids, and human derived apoE422k can be derived for other membrane forming lipids such as DOPC, POPC, DOPE, DPPC and natural lipids extracts such as SoyPC and EggPC and a mixture thereof as will be understood for a skilled person upon the reading of the present disclosure. [1]

The above ratios between lipids (membrane forming and various cationic lipids) and apoE-derived scaffold proteins can be derived for other membrane forming lipids such as DOPC, POPC, DOPE, DPPC and natural lipids extracts such as SoyPC and EggPC and a mixture thereof as will be understood for a skilled person, and can range from 40:1 to 240:1.

In some embodiments, the ratios between lipids (membrane forming and various cationic lipids) and apoA-derived scaffold proteins can be derived for other membrane forming lipids such as DOPC, DMPC, POPC, DOPE, DPPC and natural lipids extracts such as SoyPC and EggPC and a mixture thereof as will be understood for a skilled person, and can range from 20:1 to 180:1.

In some preferred embodiments, the cationic NPLs herein described have apoE422k as the scaffold protein, DMPC as membrane forming lipids and DDAB as the cationic lipid in a molar ratio from 5 to 20. In some preferred embodiments, the cationic NPLs herein described have apoE422k as the scaffold protein, DMPC as membrane forming lipids and DMTAP as the cationic lipid in a molar ratio from 10 to 30.

In other preferred embodiments, the cationic NPLs herein described have apoE422k as the scaffold protein, DMPC as membrane forming lipids and MVL5 as the cationic lipid in a molar ratio from 1 to 10. In other preferred embodiments, the cationic NPLs herein described have apoE422k as the scaffold protein, DMPC as membrane forming lipids and DMEPC as the cationic lipid in a molar ratio from 2 to 20. In other preferred embodiments, the cationic NPLs herein described have apoE422k as the scaffold protein, DMPC as membrane forming lipids and DODAP as the cationic lipid in a molar ratio from 20 to 40.

In some preferred embodiments, the cationic NPLs herein described have apoE422k as the scaffold protein, POPC as membrane forming lipids and DOTAP as the cationic lipid in a molar ratio from 1 to 10. In some preferred embodiments, the cationic NPLs herein described have apoE422k as the scaffold protein, POPC as membrane forming lipids and DMTAP as the cationic lipid in a molar ratio from 1 to 10. In some preferred embodiments, the cationic NPLs herein described have apoE422k as the scaffold protein, POPC as membrane forming lipids and DMEPC as the cationic lipid in a molar ratio from 1 to 10. In some preferred embodiments, the cationic NPLs herein described have apoE422k as the scaffold protein, POPC as membrane forming lipids and DC-cholesterol as the cationic lipid in a molar ratio from 1 to 10. In some preferred embodiments, the cationic NPLs herein described have apoE422k as the scaffold protein, POPC as membrane forming lipids and DOEPC as the cationic lipid in a molar ratio from 1 to 10. In some preferred embodiments, the cationic NPLs herein described have apoE422k as the scaffold protein, POPC as membrane forming lipids and DDAB as the cationic lipid in a molar ratio from 1 to 10.

In some preferred embodiments, the cationic NPLs herein described have apoE422k as the scaffold protein, DOPC as membrane forming lipids and DMTAP as the cationic lipid in a molar ratio from 1 to 10. In some preferred embodiments, the cationic NPLs herein described have apoE422k as the scaffold protein, DOPC as membrane forming lipids and DMEPC as the cationic lipid in a molar ratio from 1 to 10. In some preferred embodiments, the cationic NPLs herein described have apoE422k as the scaffold protein, DOPC as membrane forming lipids and DC-cholesterol as the cationic lipid in a molar ratio from 1 to 10. In some preferred embodiments, the cationic NPLs herein described have apoE422k as the scaffold protein, DOPC as membrane forming lipids and DOTAP as the cationic lipid in a molar ratio from 1 to 10. In some preferred embodiments, the cationic NPLs herein described have apoE422k as the scaffold protein, DOPC as membrane forming lipids and DOEPC as the cationic lipid in a molar ratio from 1 to 10. DOPC as membrane forming lipids and DDAB as the cationic lipid in a molar ratio from 1 to 10.

In some preferred embodiments, the cationic NPLs herein described have apoA1 as the scaffold protein, DMPC as membrane forming lipids and DDAB as the cationic lipid in a molar ratio from 5 to 20. In some preferred embodiments, the cationic NPLs herein described have apoA1 as the scaffold protein, DMPC as membrane forming lipids and DMTAP as the cationic lipid in a molar ratio from 10 to 30. In other preferred embodiments, the cationic NPLs herein described have apoA1 as the scaffold protein, DMPC as membrane forming lipids and MVL5 as the cationic lipid in a molar ratio from 5 to 20. In other preferred embodiments, the cationic NPLs herein described have apoA1 as the scaffold protein, DMPC as membrane forming lipids and DMEPC as the cationic lipid in a molar ratio from 20 to 40. In other preferred embodiments, the cationic NPLs herein described have apoA1 as the scaffold protein, DMPC as membrane forming lipids and DOTMA as the cationic lipid in a molar ratio from 5 to 20.

In some embodiments, the cationic NLPs herein described can comprise a mixture of cationic lipids at ratios and concentrations identifiable by a skilled person upon reading of the present disclosure. For example, in at least one embodiment, cationic NLPs herein described can comprise DMPC:MVL5:DODAP at ratios 3:1:1 with ApoE scaffold protein. In at least one embodiment, cationic NLPs herein described can comprise DMPC:MVL5:DODAP at ratios 2:1:2 with ApoE scaffold protein. In at least one embodiment, cationic NLPs herein described can comprise DMPC:MVL5:DODAP at ratios 3:1:1 and 20% GMO additive with ApoE scaffold protein. In another embodiment, cationic NLPs herein descried can comprise DMPC:DDAB:DOPE at ratios 70:10:20.

In embodiments, herein described cationic NLPs can be prepared with various methods resulting in the assembly of the lipid component formed by the membrane forming lipid and the cationic lipids with the scaffold protein.

In particular, in some embodiments the cationic NLP lipid component and scaffold protein component can be contacted to form an admixture for a time and under conditions allowing assembling of the NLP according to methods known or identifiable by a skilled person upon reading of the present disclosure.

For example, in some embodiments, cationic NLPs herein described can be assembled by a dialysis method, which is a self-assembly process involving detergent solubilization of lipids followed by detergent removal as described for example in [2-4] A dialysis method typically involves solubilizing the membrane lipid component in a detergent, such as sodium cholate, at detergent concentrations above the critical micelle concentration. The resulting lipid/detergent solution is then incubated to allow for dissolution of the scaffold protein and sufficient interaction between the scaffold protein and lipid mixture (e.g. for about 30 min±5 min). After the incubation period, the detergent is removed (e.g. through dialysis or rinsing with detergent binding beads). The appropriate lipid to apolipoprotein ratio that will allow for self-assembly will be understood by a skilled person upon reading of the present disclosure. In particular, the NLP typically self-assemble during the detergent removal process.

For example, in some embodiments, NLPs herein described can be assembled following a detergent-binding bead method, which is a self-assembly process involving detergent solubilization of lipids followed by detergent removal. This method typically involves solubilizing the membrane lipid component in a detergent, such as sodium cholate, at detergent concentrations above the critical micelle concentration. The resulting lipid/detergent solution is then incubated to allow for dissolution of the scaffold protein and sufficient interaction between the scaffold protein and lipid mixture (e.g. for about 30 min—with the term about indicating the time±5 min). After the incubation period, the detergent is removed by incubating with detergent binding beads. The appropriate lipid to apolipoprotein ratio that will allow for self-assembly will be understood by a skilled person upon reading of the present disclosure. In particular, the NLPs typically self-assemble during the detergent removal process. An example of a detergent commonly used to prepared apolipoprotein-lipid complexes is sodium cholate.

In some embodiments, NLPs herein described can be assembled by temperature cycling method, where an admixture of lipid component and scaffold protein component forming the NLPs is subjected to a temperature transition cycle in presence of a detergent such as the one described in [5-7] In the temperature cycle, the temperature of the admixture is raised above and below the gel crystalline transition temperature of the membrane forming lipids. In particular, in accordance with an exemplary procedure the lipid component including membrane forming lipid and cationic lipids can be added to the scaffold protein at the desired lipid to scaffold protein ratio in buffer. After thoroughly mixing the components, the solution is incubated a particular temperature for a certain amount of time. For example, the solution can be maintained at 23.8° C. for at least 2 hours (see Example 3).

In some embodiments, NLPs herein described can be assembled by an in vitro coupled transcription and translation method, where self-assembly of the NLPs can be achieved while the apolipoprotein or other scaffold protein is being translated from mRNA as described for example in [8-11]. In this process, expression system lysates are mixed with the lipid component of the NLP and plasmid DNA encoding the scaffold protein. The reaction can then be allowed to proceed until assembly occurs during apolipoprotein expression (e.g. for approximately 4-24 hrs). The apolipoprotein typically contains an affinity tag (e.g. His-tag) for subsequent purification of the self-assembled NLP from the lysate.

In general, assembly of cationic NLPs can be accomplished with a wide range of ratios of total lipids to scaffold proteins. Cationic NLPs with lipid to scaffold molar ratios of about 20:1 up to about 240:1 can be synthesized. A typical assembly with apoA1 scaffold protein uses a lipid to protein molar ratio of about 80:1 and a typical assembly with apoE uses a lipid to protein molar ratio of about 170:1 wherein the term about when referred to ratios indicates that the ratio can be ±5%.

In some embodiments, the methods and systems herein described are performed at predefined lipid protein ratio, assembly conditions and/or with the use of preselected protein component and lipid component so as to increase the yield, control the size and composition of the resulting NLP, provide an NLP of pre-determined dimensions, achieve desired functionality of the NLP, such as a certain level of loading capacity for a target drug molecule. In some embodiments, the molar ratio of lipid component to scaffold protein component is 20:1, 30:1, 40:1, 50:1, 60:1, 70:1, 80:1, 90:1, 100:1, 110:1, 120:1, 130:1, 140:1, 150:1, 160:1, 170:1, 180:1, 190:1, 200:1, 210:1, 220:1, 230:1, and 240:1. In NLPs herein described, the lipid to scaffold protein component ratio can be determined on a case by case basis in view of the experimental design as will be understood by a skilled person.

Composition of an NLP can be detected by various techniques known in the art, such as high performance liquid chromatography (HPLC), reverse phase high performance liquid chromatography (RP-HPLC), mass spectrometry, thin layer chromatography, NMR spectroscopy and elemental analysis could be used to define the composition of the particles and additional techniques identifiable by a skilled person.

In several embodiments herein described, cationic NLPs show different size, compositions, and homogeneity (see Examples 1-2). Size and compositions of the resulting NLPs can be characterized by SEC (size exclusion chromatography) traces which are used to separate out molecules in solution by their size and in some cases their molecular weights as will be understood by a skilled person.

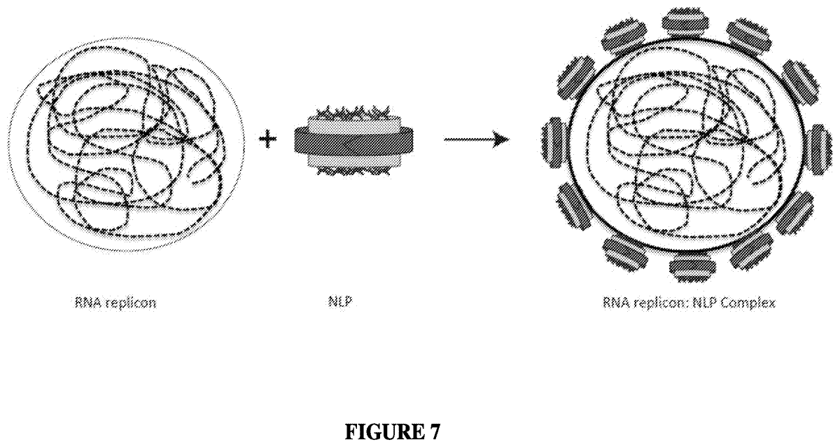

In embodiments herein described, NLPs comprising cationic lipids and optionally additional components such as polymerizable lipids, amphipathic compounds and/or target proteins and assembled as herein described, are then contacted with nucleic acids to form cationic NLPs-RNA complex which allow highly efficient in vivo delivery of the nucleic acids (see FIG. 7). Methods to include polymerizable lipids, target proteins and/or functionalized membrane forming lipids are described for example in U.S. Patent Publication No. 2009/0192299, U.S. Patent Publication No. 2009/0136937 and U.S. Pat. No. 8,883,729 issued on Nov. 11, 2014 and in U.S. Pat. No. 8,889,623 issued on Nov. 18, 2014 the content of each of which is incorporated by reference in its entirety in the present disclosure.

In some embodiments herein described cationic-NLPs can be used to form complex with one or more polynucleotides.

The term “polynucleotide” or “polynucleotide molecule” as used herein indicates an organic polymer composed of two or more monomers including nucleotides, or analogs thereof. The isoelectric point of a polynucleotide in the sense of the disclosure is less than 7 as will be understood by a skilled person. The term “nucleotide” refers to any of several compounds that consist of a ribose or deoxyribose sugar joined to a purine or pyrimidine base and to a phosphate group and that is the basic structural unit of nucleic acids. The term “nucleotide analog” refers to a nucleotide in which one or more individual atoms have been replaced with a different atom or with a different functional group. Accordingly, the term “polynucleotide” includes nucleic acids of any length, and in particular DNA, RNA, analogs and fragments thereof. A polynucleotide of three or more nucleotides is also called “nucleotidic oligomer” or “oligonucleotide.”. In particular polynucleotides in the sense of the disclosure comprise biological molecules comprising a plurality of nucleotides. Exemplary nucleic acids include deoxyribonucleic acids, ribonucleic acids, and synthetic analogues thereof, including peptide nucleic acids. Polynucleotides can typically be provided in single-stranded form or double-stranded form and in liner or circular form as will be understood by a person of ordinary skill in the art.

In the embodiments herein described, the nucleic acids that can be incorporated into the cationic NLPs include RNA, DNA, or synthetic variants that carry an overall negative charge. More specifically, the cationic NLPs can form a complex with large double-stranded DNA, such as plasmids, short double stranded DNA, short single stranded DNA, such as DNA aptamers, short single-stranded RNA, such as microRNA, short double-stranded RNA such as siRNA, adjuvant molecules such as polyl:C, long single-stranded RNA such as messenger RNA, and replicon RNA.

In some embodiments, the nucleic acids are molecules having a number of bases up to 200 bases, up to 5000 bases up to 15,000 bases, preferably from 200 to 15,000 bases, more preferably from 5,000 to 15,000 bases. In some particular embodiments, the nucleic acids herein described comprise a special type of self-amplifying RNA molecules termed replicons, which are based on alphavirus genomic RNA.

The term “alphavirus” describes enveloped single-stranded positive sense RNA viruses of the family Togaviridae. The genus alphavirus contains approximately 30 members, which can infect humans as well as other animals. Alphavirus particles typically have a 70 nm diameter, tend to be spherical or slightly pleomorphic, and have a 40 nm isometric nucleocapsid. The total genome length of alphaviruses ranges between 11,000 and 12,000 nucleotides, and has a 5′ cap and 3′ poly-A tail. There are two open reading frames (ORF's) in the genome, non-structural (ns) and structural. The ns ORF encodes proteins (nsP1-nsP4) necessary for transcription and replication of viral RNA. The structural ORF encodes three structural proteins: the core nucleocapsid protein C, and the envelope proteins P62 and E1 that associate as a heterodimer. The viral membrane-anchored surface glycoproteins are responsible for receptor recognition and entry into target cells through membrane fusion. The four ns protein genes are encoded by genes in the 5′ two-thirds of the genome, while the three structural proteins are translated from a subgenomic mRNA colinear with the 3′ one-third of the genome.

Alphaviruses can be used in gene therapy and vaccine development. Alphaviruses offers several advantages over DNA vectors in gene therapy. For example, vaccines based on DNA plasmids often contain regulatory sequences and antibiotic resistance genes. The potential integration of such sequences into the host genome by non-homologous recombination may represent an unknown risk. In contrast, replication/transcription of replicons is strictly confined to the host cell cytosol, and does not require any cDNA intermediates, nor is any recombination with or integration into the chromosomal DNA of the host required. Thus, several safety issues associated with DNA vaccines do not arise with RNA-based vaccines. Due to autonomous RNA replication, RNA replicons are also able to drive high level, cytosolic expression of recombinant antigens stimulating both the humoral and the cellular branch of the immune system.

The term “replicon” refers to a virus-based expression system based on replacement of viral structural genes by one or more genes of interest. These modified viral genomes can be synthesized in vitro and delivered into cells either by transfection or in infectious viral particles, which deliver essentially every packaged RNA molecule into the cells both in vivo and in vitro. The term replicon encompasses both DNA and RNA. Both positive- and negative-stranded viruses can be used to construct replicons and delivered as RNA, DNA, or viral replicon particles.

In some embodiments, the cationic NLPs-RNA complexes described herein comprise RNA replicons binding to a plurality of NLPs to form a cationic NLPs-replicon complex (see FIG. 7). RNA replicons herein described are derived from positive-strand RNA viruses, from which at least one gene encoding an essential structural protein has been deleted. RNA replicons can be regarded as disabled viruses unable to produce infectious progeny. Despite such gene deletions, the viral RNA can be replicated and transcribed by the viral RNA polymerase. The genetic information encoded by the replicon will be amplified many times, resulting in high levels of expression.

One or more target RNA molecules can be provided in NLPs according to methods comprising contacting the target RNA molecules with a plurality of cationic nanolipoprotein particles to provide one or more cationic-RNA nanolipoprotein particles, each comprising a target RNA molecule with its surface binding to a plurality of cationic NPLs as shown in FIG. 7. In particular, in some embodiments, to assemble cationic NLPs, a lipid mixture containing cationic lipids and membrane forming lipids can be prepared at the desired ratio. Cationic NLPs containing cationic lipids can be then prepared by using any of the methods described above that are intended for NLP assembly. Once the cationic NLPs are formed, the target RNA molecules (dissolved in a solvent) can then be added to the NLP solution and allowed to mix to allow binding of cationic NPLs (e.g. between 1 minute and 10 hours) to the outer surface of the target RNA molecule. Additional variations and embodiments of methods to incorporate a target RNA molecule in an NLP of the disclosure will be identifiable by a skilled person upon reading of the present disclosure.

In some embodiments, cationic nanolipoprotein particles when formulated with 1 μg RNA replicon demonstrated comparable transfection ability to 30 μg unformulated replicon through in vivo gene expression in mouse model.

In some embodiments, the cationic NLPs-RNA complex used to deliver the loaded RNA have a ratio of 0.05-0.2 between the number of nitrogen atoms in the cationic NLPs and the number of phosphate groups in the RNA, 10-fold lower compared to conventional approaches.

In some embodiments, cationic NLPs herein described can be used in biomedical applications, including gene therapy and vaccine and immunomodulation applications. In particular, cationic NLPs herein described can be used in nucleic acid delivery in order to achieve improved in vivo nucleic acid loading capacity and transfection efficiency. Nanoparticle-mediated RNA delivery performed with cationic NLPs herein described is expected to address several limitations of conventional gene therapy approaches due to their self-amplifying abilities, flexibility in constructing antigens with mutations, highly efficient protein synthesis, and enhanced immune protection from antigens. Additional features can also be implemented, such as adding adjuvants, functional polymers, and membrane-associated proteins for targeted in vivo delivery.

As used herein, “gene therapy” or “gene transfer” refers to the use of a nucleic acid as a drug, and to its delivery into a cell to treat a disease. Gene therapy aims to replace a faulty gene or to add a new gene in an attempt to treat a disease or to improve the subject's ability to fight against said disease. Gene therapy can be useful for treating a wide range of diseases such as genetic diseases and cancer.

According to the invention, the term “vaccine” relates to a pharmaceutical preparation (pharmaceutical composition) or product that upon administration induces an immune response. A vaccine may be used for the prevention or treatment of a disease.

The term “treatment” as used herein refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

In some embodiments, the cationic-NLP composition can be customized for desired nucleic acids loading capacity and stability by adjusting the molar ratio of the cationic NLPs and the nucleic acids of interest, the molar ratio of the total lipid component and the scaffold protein component, and the molar ratio of the cationic lipids and the membrane forming lipids.

In some embodiments, the cationic NLPs-RNA complex used for delivering nucleic acids have a lipid component to scaffold molar ratio ranging from 20:1 to 240:1, depending on the scaffold protein and the lipid component used as will be understood by a skilled person.

For example, in NLPs herein described having apoE4 and variants as scaffold protein and a lipid component comprising DMPC as the membrane forming lipid and DMTAP, the molar ratios of lipid component:scaffold protein component can range from 40:1 to 240:1, preferably from 100:1 to 240:1, even more preferably at 170:1. In NLPs herein described having apoA1 and variants as scaffold protein and a lipid component comprising DMPC as the membrane forming lipid and DMTAP, the molar ratios of lipid component:scaffold protein component can range from 40:1 to 240:1, preferably from 40:1 to 100:1, even more preferably at 80:1.

In some embodiments herein described, the cationic NLPs formed with apoA1 as the scaffold protein have a smaller diameter than those formed with apoE4 scaffold protein. For example, in one embodiment, the cationic NLP formed with apoA1 has a diameter of 13.6 nm, while the cationic NLP formed with apoE4 has a diameter of 17.6 nm. (Chromy, B. A. et al.) Different apolipoproteins impact nanolipoprotein particle formation. (Journal of the American Chemical Society 129, 14348-14354, doi:10.1021/ja074753y (2007)) (Fischer, N. O. et al.) Isolation, characterization, and stability of discretely-sized nanolipoprotein particles assembled with apolipophorin-III. PloS one 5, e11643, doi:10.1371/journal.pone.0011643 (2010).) In some embodiments, the cationic NLPs formed with apoA1 have a dimeter in a range between 6 and 14 nm. The cationic NLPs formed with ApoE have a diameter in a range between 12 and 30 nm. In both types cationic NLPs, identical lipid components can be used.

In some embodiments, the cationic NLPs used for delivering nucleic acids have a lipid component comprising membrane forming lipids in an amount from 1 to 40 mol % of the lipid component and cationic lipids in an amount from 1 to 60 mol % of the lipid component, depending on the membrane forming lipids and cationic lipids as will be understood by a skilled person. In some embodiments, the NLP herein described comprise membrane forming lipid and cationic lipids in molar ratios ranging from 99:1 to 4:6.

For example, in cationic NLPs herein described having a lipid component comprising DMPC as the membrane forming lipid and DMTAP as cationic lipids, the DMPC can have a mol percentage ranging from 40 mol % to 99 mol %, preferably from 60 mol % to 95 mol % and DMTAP can have a mol percentage ranging from 1 mol % to 60 mol %, preferably from 5 mol % to 40 mol %. In some particular embodiments, DMTAP constitutes 20 mol % of the total lipid content. (see Examples 3-8).

In several embodiments cationic-NLPs herein described can form complexes with polynucleotides of various dimensions. In particular, a cationic-NLP-polynucleotide complex herein described comprises a polynucleotide molecule attached to one or more cationic NLPs each cationic NLP comprising one or more cationic lipids and a membrane forming lipid arranged in a discoidal membrane lipid bilayer stabilized by a scaffold protein.

In particular in some of those embodiments, a cationic-NLPs can comprise 1% to 5% multivalent cationic lipids forming complexes with polynucleotides up to 200 bases, from 200 to 5000 bases, at least 5,000 bases up to 15,000 bases in size in ratios cationic-NLPs:polynucleotide of 1:1 to 1:250 or higher.

In some embodiments, cationic-NLPs comprising monovalent and/or multivalent cationic lipids can form complexes with a polynucleotide of 201 to 15,000 bases in size in various ratios as will be understood by a skilled person upon reading of the present disclosure

In some embodiments, the cationic NLPs are complexed with RNA and/or other polynucleotides in molar ratios from 10:1 to 500:1, preferably from 50:1 to 250:1, more preferably from 50:1 to 75:1. In particular, the cationic NLPs-polynucleotide complexes comprise membrane forming lipids, cationic lipids and RNA replicons in a molar ratio from 574:6:1 to 80,000:120,000:1, preferably from 3,960:40:1 to 12,000:18,000:1.

For example, in cationic NLPs-RNA complex herein described having a lipid component comprising DMPC as the membrane forming lipid and DMTAP as cationic lipids and RNA replicon. The molar ratio among DMPC, DMTAP and the RNA replicon ranges from 574:6:1 to 80,000:120,000:1, preferably from 3,960:40:1 to 12,000:18,000:1. In some preferred embodiments, the cationic NLPs-polynucleotide complex herein described comprise DMPC, DMTAP and RNA replicon at a molar ratio of 16,000:4,000:1. In some preferred embodiments, the cationic NLPs-RNA complex herein described comprise DMPC, DMTAP and RNA replicon at a molar ratio of 3,200:800:1.

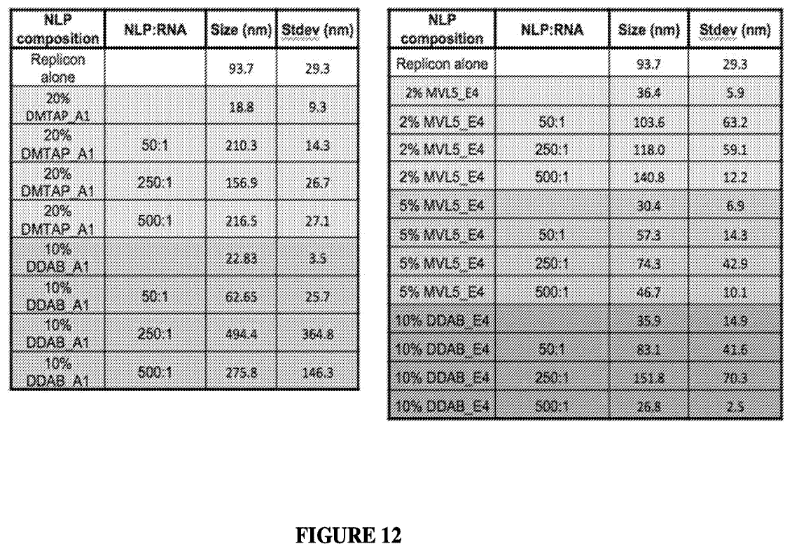

In some embodiments, the cationic NLPs have a size 13-15 nm and the RNA replicon has a size of 50 nm and the cationic NLPs-RNA complex comprising both the cationic NLPs and the RNA replicon have a size ranging from 90 nm and 558 nm, depending on the type of scaffold proteins, membrane forming lipids, and cationic lipids used in the formation of the cationic NLPs (see FIG. 12).

The cationic NLPs-RNA complex described in several embodiments here demonstrate to stay stable when stored at 4° C. for over 50 days.

In embodiments herein described, the cationic NLPs-polynucleotide complex provide an efficient, versatile and reproducible delivery of RNA replicon molecules. In particular, the cationic NLP-replicon complex formulations are stable, easily prepared and do not fully encapsulate the replicon while capable of protecting against RNase degradation, shielding access to outside molecules as well as increasing the replicon's in vivo transfection efficiency (see Examples 6-8).

In some embodiments, the cationic NLPs-RNA complex used to deliver the loaded RNA have a ratio of 0.05-0.2 between the number of nitrogen atoms in the cationic NLPs and the number of phosphate groups in the RNA, 10-fold lower compared to conventional approaches. The cationic nanolipoprotein particles herein described in several embodiments when formulated with 1 μg RNA replicon demonstrated comparable transfection ability to 30 μg unformulated replicon through in vivo gene expression in mouse model.

In some embodiments, the RNA or other polynucleotide molecules are formulated at molar ratios ranging from 10 to 500 NLPs per RNA/polynucleotide molecule. In some embodiments, apoAI NLPs are formulated with RNA/polynucleotide molecules (10,000-15,000 bases) at molar ratios ranging from 50 to 500 NLPs per RNA/polynucleotide molecules. In some preferred embodiments, apoAI NLPs are formulated with RNA/polynucleotide molecules (10,000-15,000 bases) at molar ratios ranging from 50 to 150 NLPs per RNA molecules. In some embodiments, apoE NLPs are formulated with RNA molecules (10,000-15,000 bases) at molar ratios ranging from 10 to 100 NLPs per RNA molecules. In some preferred embodiments, apoE NLPs are formulated with RNA/polynucleotide molecules (10,000-15,000 bases) at molar ratios ranging from 10 to 25 NLPs per RNA/polynucleotide molecules.

In embodiments herein described, cationic-NLPs can form stable cationic-NLP:RNA complexes comprised of different lipids, various percentages of cationic lipid, and a range of ratios of cationic-NLP added per RNA complex.

In some embodiments, cationic-NLPs herein described can comprise membrane forming lipid of Formula (XII). In those embodiments, exemplified by the representative example of DMPC, the cationic-NLPs can comprise various percentages and cationic lipid components with the lipid of Formula XV being predominant. In some cases, stability and/or homogeneity of the NLPs prepared can be controlled by selecting the membrane forming lipid and/or scaffold protein used. In particular, the ratios between membrane forming lipids and cationic lipids can be selected on the types of membrane forming lipids and cationic lipids selected for the NLP assembly. In general, DMPC is configured to have more ordered lipid-lipid interactions compared to DOPC, resulting in tighter packing of the lipids in the bilayer. Consequently, more lipids are contained in a single NLP. Therefore more cationic lipids can be subsequently packed into a DMPC-based NLPs compared to DOPC-based NLPs. As a consequence, for example, a 5% of cationic lipid incorporation can result in more positive charges in a DMPC-based NLPs than a DOPC-based NLPs as there is a greater number of lipids in the DMPC-based NLP than in the DOPC-based NLP.

In some embodiments, the specific type of cationic lipid can be selected to control the binding ability of the resulting cationic-NLP RNA complex.

In some embodiments, the cationic lipid can be a cationic lipid of Formula XVI or XVII, in those embodiments, the resulting cationic-NLPs provides tight RNA binding at low cationic lipid percentages as compared to other types of cationic-NLP RNA complexes including cationic lipids such as the one with formula (XVIII).

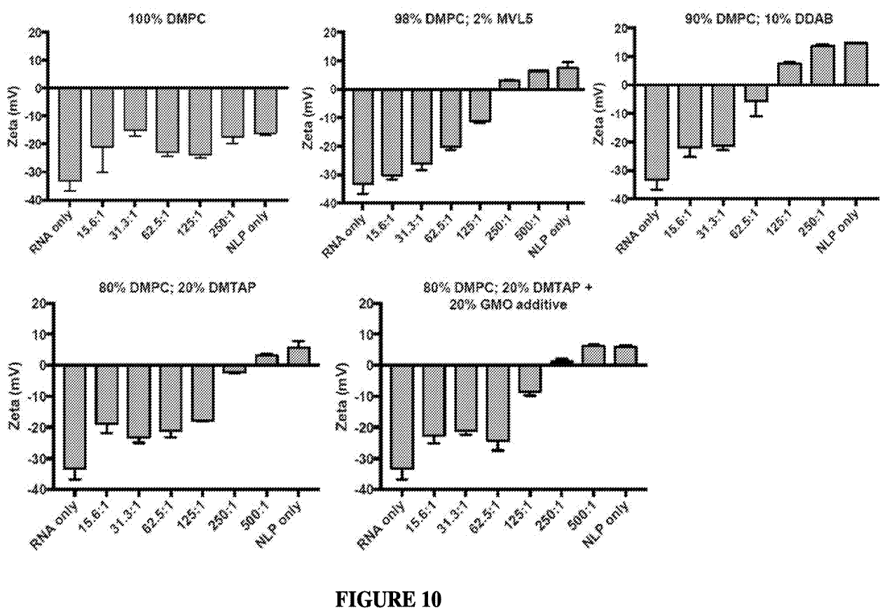

For example, cationic-NLPs made with 1%, 2%, or 5% MVL5 and 99, 98, or 95% DMPC, respectively, indicate tight RNA binding at these low cationic lipid percentages as compared to other types of cationic-NLP RNA complexes such as the ones of Formula (XVIII). For example, cationic-NLPs made with 5, 10, or 20% DDAB with 95, 90, 80% DMPC, respectively, bind to RNA at higher cationic lipid percentages than MVL5 containing cationic-NLPs (FIG. 10).

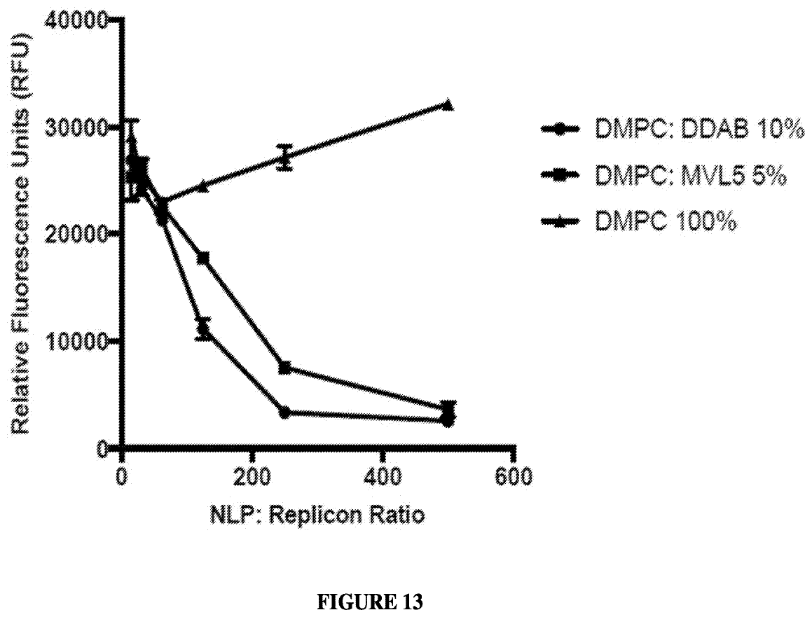

In some embodiments, the size of cationic-NLPs;RNA complex herein described can be controlled by appropriate selection of the scaffold protein of the cationic-NLPS. In particular, in the exemplary embodiment where the scaffold protein is ApoA1 the related cationic-NLPs tend to form cationic-NLP RNA complexes larger than 500 nm in diameter as the ratio of cationic-NLP to RNA increases beyond 250:1 ratio. In the exemplary embodiment where the scaffold protein is ApoE4 cationic-NLPs are not form as large of complexes as the ApoA1-cat NLP complexed with RNA (FIG. 13). In some embodiments, cationic NLPs formed with ApoA1 complexes can have a size below 100 nm. In some embodiments, cationic NLPs formed with ApoA1 can have a size larger than 100 nm and smaller than 1000 nm. In addition to the size difference, the specific ratios of cationic-NLP:RNA associated with a selection of a specific is expected to affect stability.

In some embodiments selection of the concentration of the cationic lipid in the NLPs is performed to control the overall charge of the cationic-NLPs to control the stability of the cationic-NLP RNA complex. In particular, such selection can be performed in view of the type and amount of membrane forming lipid and cationic lipids as will be understood by a skilled person upon reading of the present disclosure.

The cationic NLPs:RNA complex described herein increase endocytosis and trigger enhanced cellular uptake when in contact with the cells. Once inside the cell, the scaffold proteins can help the RNA molecule escape from endosomal digestion through a “recycling mechanism” [31] (Takahashi and Smith, PNAS, 1999; Hassan et al., JBC, 2007). Naturally, after HDL particles are taken up by endocytosis, some of the apolipoproteins are redirected to an alternative pathway to avoid being degraded in lysosomes. The spared apolipoproteins are subsequently transported back toward the cell surface to be “recycled”. This provides a possible reason to account for the enhanced in vivo transfection. In addition, the cationic NLPs also provide a good leaving group that may release the RNA quickly upon rerouting back toward the plasma membrane, allowing the RNA to refold quickly and translate with greater efficiency.

In some embodiments, the cationic NLPs herein described further comprise additives including membrane associated proteins, amphiphilic polymers, and immunogenic adjuvants such as monophosphoryl lipid A (MPLA) or CpG oligodeoxynucleotides (CpG). These additives can be added during the assembly of the NLP herein describe also comprising one or more cationic lipids. The addition of additives display a great potential for NLP use a drug delivery vehicle [30, 33, 34]. Additional exemplary additives also include cholesterol, CpG-cholesterol, lyso lipids, monoolein (GMO), Tween 20 and Z3-14, as shown in Table 1. In particular, a variety of lipid or non-lipid additives can be added to the cationic NLP assembly at different percentages, including small molecule surfactants, single chain lipids, amphiphilic polymers and immunogenic adjuvant molecules. In general, the additive is a hydrophobic or amphipathic molecule that can incorporate fully or partially into the lipid bilayer.

In some embodiments, the cationic NLPs comprise membrane forming lipids, cationic lipids and additives, whereby the additives are present at a mol % ranging from 1 to 60, relative to total lipid (membrane forming and cationic lipids). In some embodiments, the GMO is used as the additive, and the molar ratio of the GMO with respect to the total lipid component comprising the membrane forming lipids and cationic lipids is 1:99 to 60:40. In some particular embodiments, the cationic NLPs-RNA complexes comprise DMPC as membrane forming lipids, DMTAP as cationic lipids, GMO as additives and RNA replicon at a molar ratio from 2,400:800:800:1 to 12,000:4,000:4,000:1, preferably at 2,400:800:800:1. In some embodiments, poly-arginine containing a lipidic tail is used as the additive, and the molar ratio of the poly-arginine with respect to the total lipid component comprising the membrane forming lipid and the cationic lipid is 1:99 to 1:9. In some particular embodiments, the cationic NLPs-RNA complexes comprise DMPC as membrane forming lipids, DMTAP as cationic lipids, GMO as additives and RNA replicon at a molar ratio from 3,120:800:80:1 to 15680:4,000:320:1.

In some embodiments, the addition of additive molecules allows fine-tuning the NLP to RNA replicon binding as well as replicon in vivo transfection efficiency, such as to enhance the NLP-based RNA delivery and release, as shown in Example 8 of the current disclosure. In particular, the introduction of GMO into the cationic NLPs is able to reduce the total amount of lipids used in the NLPs from a NLP:RNA ratio of 250:1 to 50:1 while still retaining comparable in vivo transfection efficiency.

In some embodiments, functionalized lipid additives can also reinforce/stabilize the cationic-NLP. Several functionalized lipids, including DSPE PEG 2000 Azide, 16:0 Azido Cap PE, and poly-arginine containing a lipidic tail can successfully be incorporated into the cationic-NLP (LLNL slide deck-30 and 36). Other additives, including cholesterol, CpG-Cholesterol, lyso lipid, monoolein (GMO), Tween 20, and Z3-14 can all be incorporated into the cationic-NLP and form stable particles.

In some embodiments, the cationic NLPs herein described further comprise one or more telodendrimers to form telo-cationic-NLPs. The term “telodcendrimer” refers to a dendrimer containing a hydrophilic. PEG segment and one or more chemical moieties covalently bonded to one or more end groups of the dendrimer. These moieties can include hydrophobic groups, hydrophilic groups, amphiphilic compounds, and drugs. Different moieties may be selectively installed at desired end groups using orthogonal protecting group strategies.

The terms “dendrimer” and “dendritic polymer” refer to branched polymers containing a focal point, a plurality of branched monomer units, and a plurality of end groups. The monomers are linked together to form arms (or “dendrons”) extending from the focal point and terminating at the end groups. The focal point of the dendritic polymer can be attached to other segments of the telodendrimers, and the end groups may be further functionalized with additional chemical moieties.

The terms “monomer” and “monomer unit” refer to repeating units that make up the dendrons of the dendritic polymers disclosed herein. The monomers can be AB2-type monomers, where A and B are two different functional groups capable of reacting together such that the resulting polymer chain has a branch point where an A-B bond is formed. Exemplary monomers include a diamino carboxylic acid, a dihydroxy carboxylic acid and a hydroxyl amino carboxylic acid. Examples of diamino carboxylic acid groups include 2,3-diamino propanoic acid, 2,4-diaminobutanoic acid, 2,5-diaminopentanoic acid (ornithine), 2,6-diaminohexanoic acid (lysine), (2-Aminoethyl)-cysteine, 3-amino-2-aminomethyl propanoic acid, 3-amino-2-aminomethyl-2-methyl propanoic acid, 4-amino-2-(2-aminoethyl)butyric acid and 5-amino-2-(3-aminopropyl)pentanoic acid. Examples of dihydroxy carboxylic acid groups include glyceric acid, 2,4-dihydroxybutyric acid, and 2,2-bis(hydroxymethyl)propionic acid. Examples of hydroxyl amino carboxylic acids include serine and homoserine. One of skill in the art will appreciate other monomer units useful in the current disclosure.

In embodiments herein described, telo-cationic-NLPs can be prepared using similar procedures previously described for the assembly of cationic-NLPs. In particular, the cationic lipids, membrane forming lipids, telodendrimers and scaffold protein component can be mixed to form an admixture for a time and under conditions allowing assembling of the telo-cationic-NLPs according to the methods above described.

For example, in some embodiments, telo-cationic NLPs herein described can be prepared by adding the telodendrimers to the assembly mixture containing the solubilized lipids and scaffold proteins prior to surfactant removal and allowing the incorporation of the telodendrimers into NLPs, followed by detergent removal as described for example in in[2-4].

In general, assembly of telo-cationic NLPs can be accomplished with a wide range of ratios of total membrane forming lipids to scaffold proteins as previously described. Telodendrimer can be incorporated in amounts ranging from 0.1 to 10% of the lipid component, with a preferred amount between 0.1 and 2%.

In some embodiments, the telodendrimers comprise 5KCA8 or cys-5KCA8 in amounts from 0.1% to about 1% (±0.01%). In some particular embodiments, the telodendrimers such as 5KCA8 or cys-5KCA8 can be incorporated into cationic NLPs comprising scaffold protein apoA1, 80% membrane forming lipids DMPC and 20% cationic lipid DMTAP (see Example 9). In these embodiments, the cationic NLPs can further comprise 20% GMO as additives in addition to the total lipid component. In some other embodiments, the telodendrimers such as 5KCA8 or cys-5KCA8 can be incorporated into cationic NLPs comprising scaffold protein apoA1, 90% membrane forming protein DMPC and 10% cationic lipid DDAB, cationic NLPs comprising scaffold protein apoE4, 70% membrane forming lipid DMPC and 30% cationic lipid DODAP, cationic NLPs comprising scaffold protein apoE4, 98% membrane forming lipid DMPC and 2% cationic lipid MVL5, cationic NLPs comprising scaffold protein apoE4, 95% membrane forming lipid DMPC and 5% cationic lipid MVL5, cationic NLPs comprising scaffold protein apoA1, 60% membrane forming lipid DMPC and 40% cationic lipid DMEPC, and cationic NLPs comprising scaffold protein apoA1, 60% membrane forming lipid DMPC, 20% cationic lipid DOPE and 20% cationic lipid DODAP.

Benefits of further incorporating telodendrimer into the cationic NLPs include reducing NLP-NLP aggregation, providing a way to fine-tune the size of the NLPs, and providing a chemical moiety that is amenable to conjugation or modification. In addition, PEgylation of nanoparticle or nanoparticle: RNA complex can confer stealth and provide increased stability and less susceptibility in vivo.

In some embodiments, the cationic-NLPs and/or cationic-NLPs polynucleotide complexes can be comprised in a composition together with a suitable vehicle. The term “vehicle” as used herein indicates any of various media acting usually as solvents, carriers, binders or diluents for the cationic-NLPs and/or cationic-NLPs polynucleotide complexes are comprised in the composition as an active ingredient. In particular, the composition including the multi-cationic-NLPs and/or cationic-NLPs polynucleotide complexes can be used in one of the methods or systems herein described.

In some embodiments, an NLP can be included in pharmaceutical compositions (e.g. a vaccine) together with an excipient or diluent. In particular, in some embodiments, pharmaceutical compositions are disclosed which contain NLP, in combination with one or more compatible and pharmaceutically acceptable vehicle, and in particular with pharmaceutically acceptable diluents or excipients. By “pharmaceutically acceptable” it is meant the carrier, diluent or excipient must be compatible with the other ingredients of the formulation and not deleterious to the recipient thereof.

The term “excipient” as used herein indicates an inactive substance used as a carrier for the active ingredients of a medication. Suitable excipients for the pharmaceutical compositions herein disclosed include any substance that enhances the ability of the body of an individual to absorb the NLP. Suitable excipients also include any substance that can be used to bulk up formulations with NLP to allow for convenient and accurate dosage. In addition to their use in the single-dosage quantity, excipients can be used in the manufacturing process to aid in the handling of NLP. Depending on the route of administration, and form of medication, different excipients may be used. Exemplary excipients include but are not limited to antiadherents, binders, coatings disintegrants, fillers, flavors (such as sweeteners) and colors, glidants, lubricants, preservatives, sorbents.

The term “diluent” as used herein indicates a diluting agent which is issued to dilute or carry an active ingredient of a composition. Suitable diluent include any substance that can decrease the viscosity of a medicinal preparation.

In certain embodiments, compositions and, in particular, pharmaceutical compositions can be formulated for systemic administration, which includes parenteral administration and more particularly intravenous, intradermic, and intramuscular administration. In some embodiments, compositions and, in particular, pharmaceutical compositions can be formulated for non-parenteral administration and more particularly intranasal, intratracheal, vaginal, oral, and sublingual administration.

The terms “administration of” and or “administering” should be understood to mean providing a pharmaceutical composition in a therapeutically effective amount to the subject in need of treatment. The terms “therapeutically effective amount” refer to the amount of the subject compound that will elicit the biological or medical response of a tissue, system, animal or human.

The term “subject” as used herein refers to any individual or patient to which the subject methods are performed. Generally the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

Exemplary compositions for parenteral administration include but are not limited to sterile aqueous solutions, injectable solutions or suspensions including NLP. In some embodiments, a composition for parenteral administration can be prepared at the time of use by dissolving a powdered composition, previously prepared in a freeze-dried lyophilized form, in a biologically compatible aqueous liquid (distilled water, physiological solution or other aqueous solution).

The term “lyophilization” (also known as freeze-drying or cryodesiccation) indicates a dehydration process typically used to preserve a perishable material or make the material more convenient for transport. Freeze-drying works by freezing the material and then reducing the surrounding pressure and adding enough heat to allow the frozen water in the material to sublime directly from the solid phase to gas.

If a freeze-dried substance is sealed to prevent the reabsorption of moisture, the substance may be stored at room temperature without refrigeration, and be protected against spoilage for many years. Preservation is possible because the greatly reduced water content inhibits the action of microorganisms and enzymes that would normally spoil or degrade the substance.

Lyophilization can also cause less damage to the substance than other dehydration methods using higher temperatures. Freeze-drying does not usually cause shrinkage or toughening of the material being dried. In addition, flavours and smells generally remain unchanged, making the process popular for preserving food. However, water is not the only chemical capable of sublimation, and the loss of other volatile compounds such as acetic acid (vinegar) and alcohols can yield undesirable results.

Freeze-dried products can be rehydrated (reconstituted) much more quickly and easily because the process leaves microscopic pores. The pores are created by the ice crystals that sublimate, leaving gaps or pores in their place. This is especially important when it comes to pharmaceutical uses. Lyophilization can also be used to increase the shelf life of some pharmaceuticals for many years.

In pharmaceutical applications freeze-drying is often used to increase the shelf life of products, such as vaccines and other injectables. By removing the water from the material and sealing the material in a vial, the material can be easily stored, shipped, and later reconstituted to its original form for injection

In some embodiments, cationic-NLPs herein described can be used to deliver one or more polynucleotide molecules to target environment is described. The method comprises contacting the target environment with one or more cationic-NLPs-polynucleotide complexes herein described comprising the one or more polynucleotide molecules.

In some embodiments, cationic-NLPs herein described methods and systems for delivery of polynucleotides to facilitate gene expression of therapeutic proteins (non-vaccine applications).

In some embodiments, cationic-NLPs herein described can be used to in methods and systems to perform an assay on polynucleotides loaded in the cationic-NLP-nucleotide complexes of the present disclosure

According to additional aspects, cationic-NLPs and related complexes can be used in methods and systems, comprising forming and using the cationic nanolipoprotein particles herein described are also provided in the present disclosure.

In some embodiments, cationic NLPs herein described and related components can be provided as a part of systems in accordance to various embodiments herein described.

In some embodiments, the systems herein described can be provided in the form of kits of parts. In a kit of parts, membrane forming lipid and cationic lipids can be provided in various combinations with one or more functionalized amphipathic compounds, one or more membrane protein, one or more additives, one or more telo-dendrimers, and/or scaffold proteins or fragments thereof. In the kits of parts, the components can be comprised in the kit independently possibly included in a composition together with suitable vehicle carrier or auxiliary agents.

Additional components can also be included and comprise, reference standards, and additional components identifiable by a skilled person upon reading of the present disclosure.

In the kit of parts herein disclosed, the components of the kit can be provided, with suitable instructions and other necessary reagents, in order to perform the methods here disclosed. In some embodiments, the kit can contain the compositions in separate containers. Instructions, for example written or audio instructions, on paper or electronic support such as tapes or CD-ROMs, for carrying out the methods herein described, can also be included in the kit. The kit can also contain, depending on the particular method used, other packaged reagents and materials (i.e. wash buffers and the like).

In some embodiments, a method of delivering a polynucleotide to a target environment comprising contacting the target environment with a cationic-nanolipoprotein (NLPs)-polynucleotide complex is provided. In specific embodiments, the target environment in a cell or a tissue. In other embodiments, a method of delivering a polynucleotide to a subject comprising administering to the subject a cationic nanolipoprotein (NLPs)-polynucleotide complex is provided. A method of inducing expression of a protein in a subject comprising administering to the subject a cationic nanolipoprotein (NLPs)-polynucleotide complex, is also provided. Further, a method of stimulating a humoral and a cellular immune response in a subject comprising administering to the subject a cationic nanolipoprotein (NLPs)-polynucleotide complex is provided. In specific embodiments, the cationic NLPs polynucleotide complex comprises one or more cationic lipids and a membrane forming lipid arranged in a discoidal membrane lipid bilayer stabilized by a scaffold protein attached to a polynucleotide, the polynucleotide is an RNA replicon, and the cationic NLPs polynucleotide complex further comprises an adjuvant, a functional polymer, a membrane-associated protein for targeted delivery, or a combination thereof. In specific embodiments, the subject is human.

In some embodiments, a pharmaceutical composition comprising a nanolipoprotein or a cationic-nanolipoprotein-polynucleotide complex and a pharmaceutically acceptable vehicle is provided.

Further details concerning the identification of the suitable carrier agent or auxiliary agent of the compositions, and generally manufacturing and packaging of the kit, can be identified by the person skilled in the art upon reading of the present disclosure.

### EXAMPLES

The methods and system herein disclosed are further illustrated in the following examples, which are provided by way of illustration and are not intended to be limiting. In particular, cationic NLPs comprising various membrane forming lipids, cationic lipids, additives, telo-dendrimers alone or together with RNA replicon to form NLP-RNA complex were prepared and characterized in vitro and in vivo. A skilled person will be able to use other membrane forming lipids, cationic lipids, additives, telo-dendrimers, scaffold proteins and RNA molecules herein described. The following materials and methods were used

Protein expression and purification: The expression clone for the 22 kDa N-terminal fragment of human apolipoprotein E4 (apoE422k, kindly provided by Dr. Karl Weisgraber) featuring a cleavable His-tag [61] was expressed and purified as previously described. [5, 62]. ApoAI featured an N-terminal His-tag and was expressed and purified as described for apoE4 above. However, since the apoAI expression product was not a fusion protein, thrombin-based cleavage or subsequent gel purification were not required.

Cationic NLP synthesis NLPs were synthesized according to a previously described in vitro assembly method [1] with slight modifications. Briefly, bulk lipid and cationic lipid(s) of choice dissolved in chloroform were formulated at various molar percentages. Chloroform was then removed using a continuous stream of N₂ gas and further dried via vacuum. Lipids were re-dissolved in PBS buffer containing 30 mM cholate and incubated at 20-25° C. for 1 hour, shaking at 1400 rpm. Scaffold proteins ApoA1 (80:1), ApoE3 (170:1) and ApoE4 (170:1) scaffold protein:lipid molar ratios were then added to the lipid/cholate mixture and incubated, shaking, for another hour. Subsequently, bio beads (Bio-Rad) were added and left shaking overnight 20-25° C. at 1100 rpm to remove the cholate. Biobeads were then filtered and NLP filtrate was run on SEC to analyze NLP formation.

Preparation of Replicon RNA Replicon RNA was acquired from SGVI and was synthesized using previously published methods (B. Bosworth, M. M. Erdman, D. L. Stine, I. Harris, C. Irwin, M. Jens, et al. Replicon particle vaccine protects swine against influenza. Comp Immunol Microbiol Infect Dis, 33 (6) (2010))

Preparation of RNA/NLP formulation Replicon was formulated with NLP at various NLP:Replicon (N/R) molar ratios. Replicon was prepared at 20 μg/ml in PBS for most formulations, but varied from 2 μg/ml to 600 μg/ml. To make formulations, the order of addition was PBS, replicon, and NLP. The formulations were incubated at room temperature for at least 5 minutes, then were subsequently used for further characterization or mouse injection.

RNase protection assay Naked RNA and NLP:Replicon complex were exposed to 3.8 mAU of RNase A (Life Technologies) per μg RNA for 1 minute at room temperature. RNase was then inactivated with the addition of 2 AU of Anti-RNase (Life Technologies) per 1 ng RNase at room temperature for 30 minutes. Afterwards, 1% Triton was added to each sample to disrupt NLP and release RNA. A 1% agarose TAE gel was prepared and loaded. 0.1 μg RNA was loaded per lane, and the gel was run at 90V for 30 minutes. Gel was then stained with SYBR Gold (Invitrogen; 1:1000 dilution in water) for 1 hour while shaking. Gel was imaged with Kodak Gel Logic 200 with Trans UV.

Dye exclusion analysis NLP:Replicon complexes were formulated at various molar ratios. In short, PBS, RNA, then NLP were mixed together and allowed to incubate at room temperature for at least 5 minutes. 0.1 μg RNA was added to each vial. Dye intercalation was visualized via Quant-iT Ribogreen RNA Reagent and Kit (Invitrogen) according to manufacturer's instructions.

RT-PCR quantitation Naked RNA and NLP:Replicon complexes were prepared as described in “RNase protection assay” for RNase and Anti-RNAse treatment. QuantiTect Probe RT-PCR kit (Qiagen) was used to run samples, adding 1% Triton to the Master Mix. 5 ng RNA was loaded per PCR well. PCR was run at 30° C. for 30 minutes, 95° C. for 15 minutes, then [95° C. for 15 seconds, 60° C. for 30 seconds)×30 cycles] (7900 HT Fast Real-Time PCR Machine; Applied Biosystems/Life Technologies). Crossing threshold (Ct) analysis was performed on the resulting amplification curves.

Zeta potential analysis The Zeta potential and particle size of the NLP and NLP-Replicon complex were measured using a Zetasizer Nano ZS 90 (Malvern Instruments, Orsay, France). NLP or NLP-Replicon samples were diluted in 1 ml water solution containing 4% PBS. Zeta potential and size distribution were analyzed at 25° C. according to the manufacturer's instructions. Particle sizes are reported as Number Mean (d.nm) with the polydispersity index (pdi).

In vivo transfection and evaluation All the mice were obtained from Envigo and housed at Lawrence Livermore National Laboratory Animal Care Facility. The experiments were approved and conducted according to the IACUC. All mice were female BALB/c, aged 6-10 weeks and weighing between 15 and 25 g. Mice were bilaterally injected with unformulated RNA or NLP formulated RNA complexes in quadriceps. After 4 to 5 days, mice were imaged at UC Davis Center for Molecular and Genomic Imaging (CMGI). Five minutes before imaging, mice were injected intraperitoneally with 150 mg/kg of luciferin solution (Caliper Lifesciences). Mice were then anesthetized [2% (vol/vol) isoflurane in oxygen] and transferred to the IVIS Spectrum instrument (Perkin Elmer). All images were quantified based on the total flux (photons per second) and all parameters were kept constant across experiments.

### Example 1: High Throughput Synthesis of Cationic-NLPS

Truncated versions of mouse apolipoproteins, apoA1-Δ49 and apoE4-22k as candidate scaffold proteins were used for the cationic-NLP synthesis (respectively referred to apoA1 and ApoE4). For the NLP zwitterionic bulk lipids, DOPC, POPC and DMPC were used; all of which have shown stable NLP formation [2] [3] [4]. Also, a wide range of cationic lipids and additives at different percentages were tested to verify the compatibility with the scaffold protein and bulk lipid of interest (Table 1). Table 1 lists various scaffold proteins, bulk lipids, cationic lipids, and other additives tested for high throughput cationic-NLP synthesis.

TABLE 1
High Throughput cationic-NLP Synthesis.

| Scaffold | Zwitterion | | Other |
|---|---|---|---|
| Proteins | Bulk Lipids | Cationic Lipids | Additives |
| ApoA1 | DMPC | DC-Cholesterol | Cholesterol |
| ApoE4 | DOPC | DDAB | CpG- |
| | POPC | DMEPC | Cholesterol |
| | | DMTAP | Lyso Lipid |
| | | DODAP | Monoolein |
| | | DOEPC | (GMO) |
| | | DOPE | Tween 20 |
| | | DOTAP | Z3-14 |
| | | DOTMA | |
| | | MVL5 | |

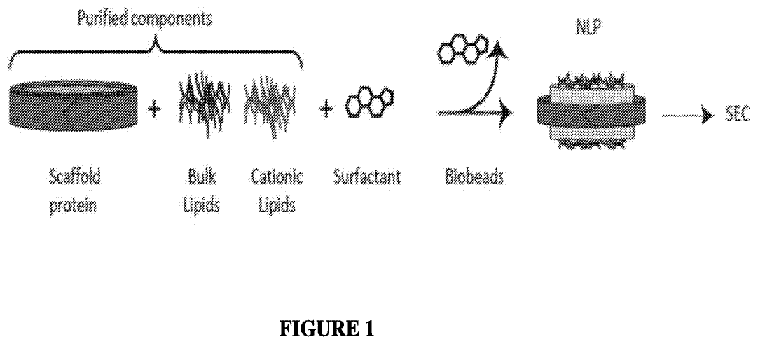

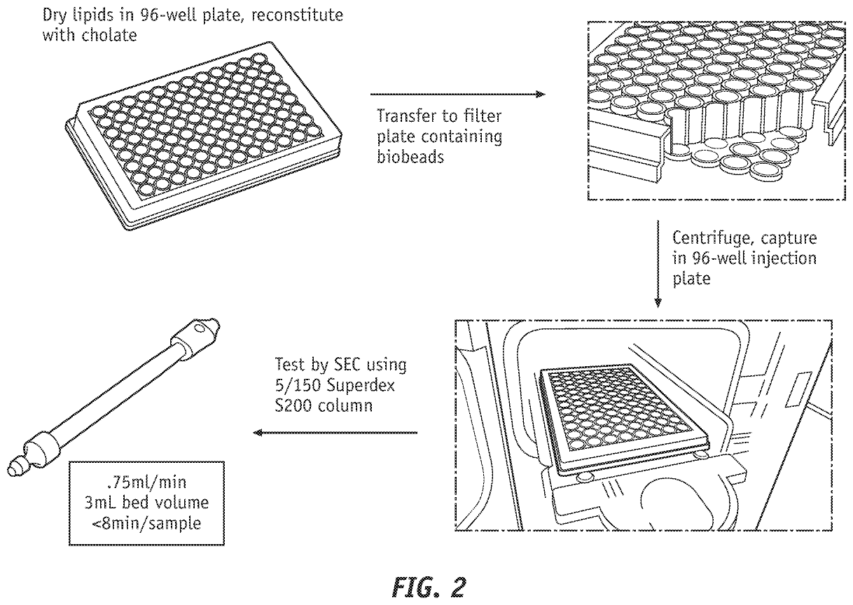

For high throughput screening, the cationic-NLPs were synthesized on a 96 well plate as shown schematically in FIG. 1 and FIG. 2 and were directly injected onto size exclusion chromatography (SEC) to access for proper NLP formation.

### Example 2: Determination of Formation of Cationic-NLPS by Analytical Size Exclusion Chromatography (SEC)

The assembly of cationic-NLPs synthesized according to the procedure described in Example 1 was evaluated using a High Throughput Screening (HTS) approach illustrated in FIG. 2.

NLPs were synthesized according to a previously described in vitro assembly method with slight modifications (Fischer, N. O. et al. Evaluation of nanolipoprotein particles (NLPs) as an in vivo delivery platform. PLoS One 9 (2014)). Briefly, bulk lipid and cationic lipid(s) of choice dissolved in chloroform were formulated at various molar percentages in a round bottom polypropylene 96 well plate. Chloroform was then removed using a continuous stream of N2 gas and further dried via vacuum. Lipids were re-dissolved in PBS buffer containing 30 mM cholate and incubated at 20-25° C. for 1 hour, shaking at 1000 rpm. Scaffold proteins ApoA1 (80:1) and ApoE (170:1) scaffold protein:lipid molar ratios were then added to the lipid/cholate mixture and incubated, shaking at 1000 rpm for another hour. Subsequently, lipid protein mixture was transferred to a 96 well Durapore membrane filter plate (Millipore). Bio beads (Bio-Rad) were added to wells and left at 20-25° C. for 1 hour, shaking at 800 rpm to remove the cholate. Biobeads were then filtered by centrifuging the plate at 1,000 rpm for 1 minute. NLP filtrate was collected in a Shimadzu conical bottom shallow 96 well plate. Plate was then covered with Shimadzu Teflon/silicone mat. NLP filtrate was run on SEC to analyze NLP formation.

The SEC traces from the high throughput NLP synthesis were numerically scored 0 to 2 based on the peak height and size of NLP formed, homogeneity of the NLP population, as well as the percentage of large aggregates and remaining free proteins.

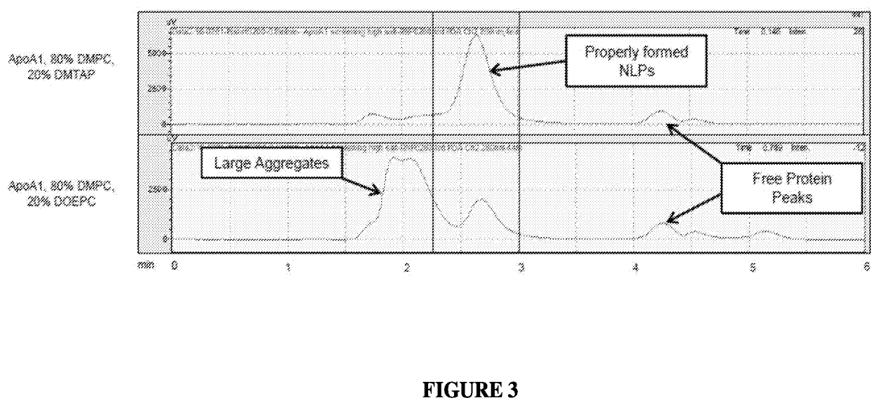

FIG. 3 shows exemplary SEC results of successful formation of cationic NLP, that received a score of 2 (upper panel), and poor NLP formation, that received a score of 0 (lower panel). The top SEC trace is an example of successful cationic NLP formation, with scaffold protein apoA1, 80% DMPC bulk lipid, and 20% DMTAP cationic lipid. It received a score of 2. The bottom SEC trace shows poor NLP formation with scaffold protein apoA1, 80% DMPC bulk lipid, and 20% DOEPC cationic lipid. It received a score of 0.

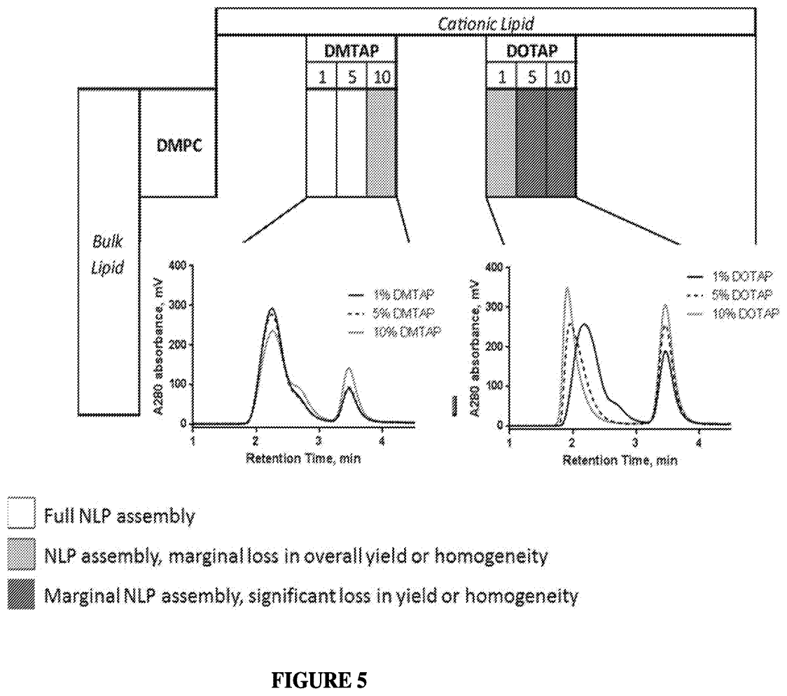

Stable cationic NLP formation is dependent on the type of scaffold protein, lipid identity, and molar ratios of all assembly constituents. The type of cationic lipid has profound impact on NLP formation. Various combinations of scaffold proteins, membrane forming lipids (bulk lipids), and cationic lipids at different ratios were tested. The results are shown in FIGS. 5-6.

FIG. 6 shows a diagram illustrating a score chart of results of High Throughput testing of exemplary cationic-NLPs using SEC analysis. Several bulk lipids (DMPC, POPC, or DOPC), scaffold proteins (ApoA1 or ApoE4) and cationic lipids were tested and assessed for proper cationic-NLP formation. Cationic-NLPs comprising cationic lipids DC-cholesterol, DDAB, DMEPC, DMTAP, DOEPC, DOTAP, or MVL5, at percentages of 1%, 5%, 10%, 15%, 20%, 30% or 40% were tested. The detected assembly for each sample is shown in a score chart wherein full cationic-NLP assembly was given a score of 2 (dark gray bar), cationic-NLPs assembly with margin loss in overall yield or homogeneity was given a score of 1 (light gray bar), and marginal cationic-NLP assembly with significant loss in yield or homogeneity was given a score of 0 (black bar), which are separately indicated. If the combination was not tested, it is left blank. This is a representative depiction of the cationic-NLPs tested.

FIG. 5 demonstrates how the quality of cationic-NLPs is assessed by size exclusion chromatography. The two chromatograms illustrate exemplary results of SEC performed on cationic-NLPs prepared with apoE comprising 1% DMTAP (black), 5% DMTAP (red), and 10% DMTAP (blue) (left panel) and on cationic-NLPs comprising 1% DOTAP (black), 5% DOTAP (red), and 10% DOTAP (blue) (right panel). Large non-NLP structures exhibit retention times less than 2 minutes. NLPs exhibit retention times between 2 and 2.7 minutes. Unincorporated scaffold protein exhibits retention times between 3.2 and 3.8 minutes. In the illustration the detected assembly for each sample is shown in a score chart wherein full cationic-NLP assembly (green bar), cationic-NLPs assembly with margin loss in overall yield or homogeneity (yellow) and marginal cationic-NLP assembly with significant loss in yield or homogeneity (red bar) are separately indicated.

The results shown in FIGS. 5-6 indicate that both apoA1 and apoE4 can form cationic NLP. Among the three membrane forming lipids tested, DMPC tolerated higher percentages of cationic lipid than POPC or DOPC. When DMPC is the bulk lipid, DMEPC, DODAP and DMTAP are among the most tolerated cationic lipids, and can comprise up to 40% of the total lipid. Other cationic lipids such as DDAB and MVL5 are well tolerated at up to 20%. It was also demonstrated that a variety of lipid or non-lipid additives can be added to the cationic NLP assembly, including small molecule surfactants, single chain lipids, amphiphilic polymers and immunogenic adjuvant molecules. Other factors like lipid to protein ratio and total lipid concentration in the assembly may also impact cationic NLP formation.

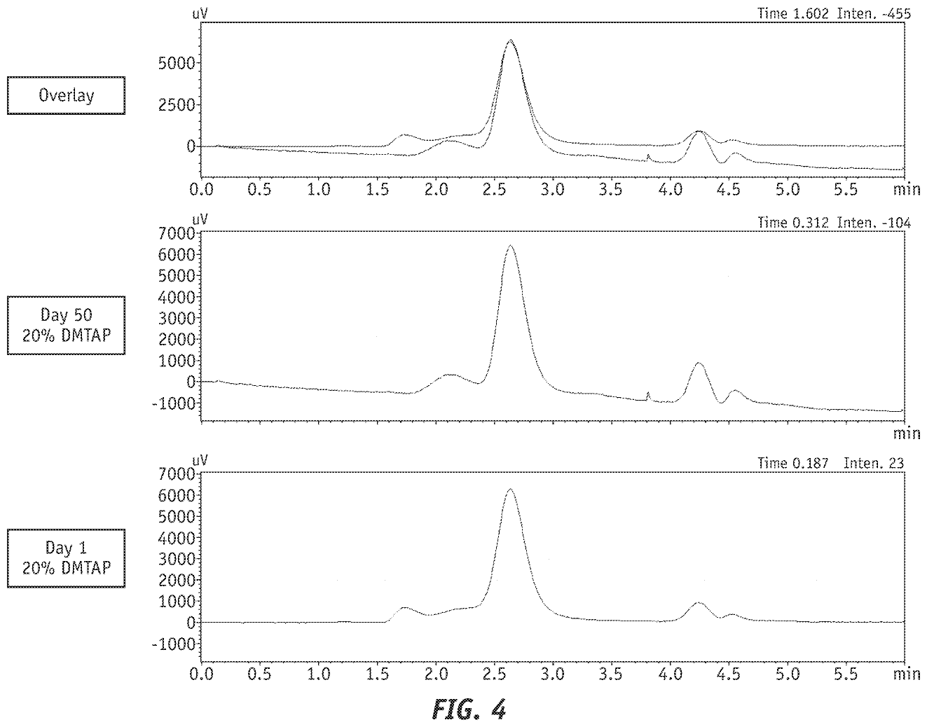

SEC analysis was also used to assess cationic NLP stability. In FIG. 4, 80:20 DMPC:DMTAP NLPs were injected onto SEC on day 1 to assess for proper NLP formation (results shown in bottom panel). After 50 days, the same sample was injected onto SEC after being stored at 4° C. (results shown in middle panel). An overlay of both results is shown in the top panel. NLPs shown are formed with apoA1 scaffold protein. The results show that the cationic NLPs display the same size distribution as neutral NLPs and are stable when stored at 4° C. for 50 days.

### Example 3: Gel Migration/Retardation In Vitro Assessment of Cationic-NLP:Replicon Complexes

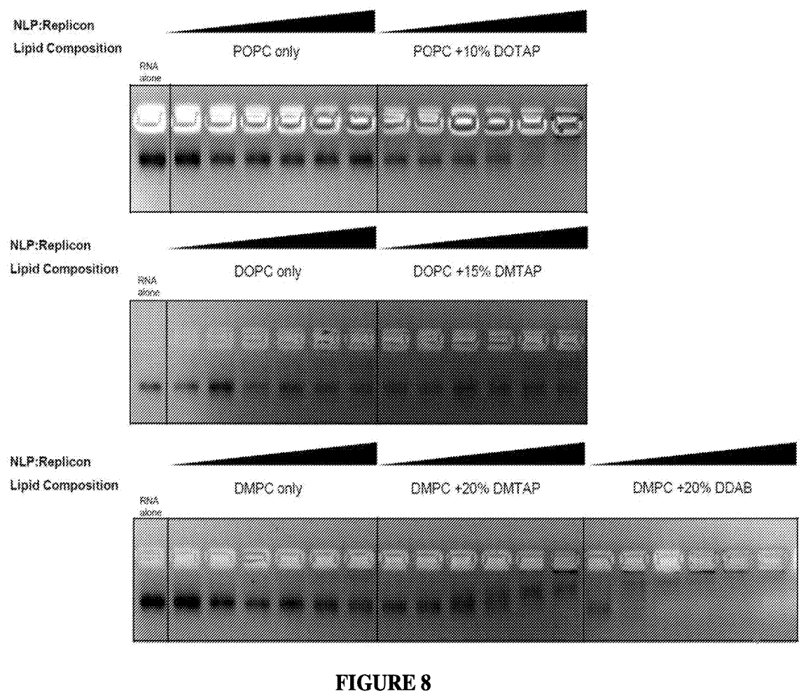

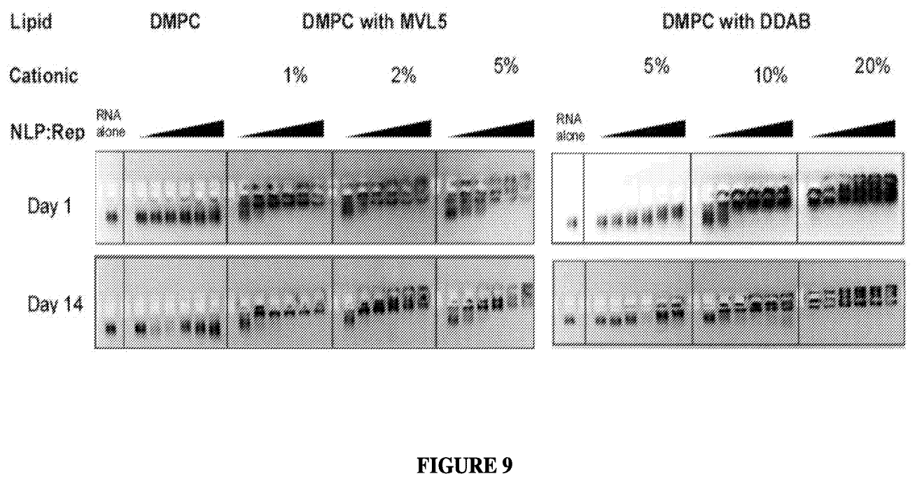

The cationic NLPs with high scores were selected and further characterized for the ability to bind to replicon. These cationic NLPs were synthesized in a scaled-up reaction and purified by SEC. After purification, the cationic NLPs were mixed with replicon at various molar ratios. Subsequently, the cationic NLP-replicon association was assessed by the migration of replicon on agarose gel. The results are shown in the gel electrophoresis images in FIGS. 8 and 9, in which relative migration of RNA based on varying NLP:RNA molar ratios are shown.

In FIG. 8, various bulk lipids (POPC, DOPC, or DMPC) are shown with several types of cationic lipids (DOTAP, DMTAP, or DDAB) at the percentages indicated. Each group shows increasing NLP:RNA molar ratios. NLP:RNA ratios are 15:1, 31:1, 62.5:1, 125:1, 250:1, and 500:1, respectively. All NLPs shown were formed with apoE4 scaffold protein. 0.1 μg RNA loaded in each well. RNA alone is shown as a control.

In FIG. 9, results for bulk lipid DMPC, or DMPC with cationic lipid MVL5 (1, 2, or 5%), or DMPC with cationic lipid DDAB (5, 10, or 20%) are shown. Each group shows increasing NLP:RNA molar ratios. NLP:RNA ratios are 15:1, 31:1, 62.5:1, 125:1, 250:1, and 500:1, respectively. Results are shown for NLP:RNA complexes either on day 1 or after 14 days stored at 4° C. All NLPs shown were formed with apoE4 scaffold protein. 0.1 μg RNA loaded per well. RNA alone is shown as a control.

The results indicate that RNA migration on the agarose gel was measurably retarded upon its binding with the cationic NLPs, but not with neutrally charged NLPs. The relative migration also correlated with the percentage of cationic lipid in the NLP, as well as the ratio of cationic NLP to replicon. In short, the increased amount of cationic charged NLPs and higher NLP to replicon ratio slows down RNA migration. In some cases, the RNA failed to migrate out of the well, indicating either full charge attenuation or the formation of significantly large structures.

Experiments were also performed to demonstrate that these NLP:Replicon complexes are stable when stored at 4° C. over a span of two weeks (data not shown).

### Example 4: Zeta Potential In Vitro Assessment of Cationic-NLP:Replicon Complexes

Based on the ability to bind to RNA replicon, a selection of cationic-NLPs were further characterized to determine the zeta potential change of RNA replicon upon cationic-NLP binding at different molar ratios. It is noted that unformulated RNA has a zeta potential of 30 mV, whereas the cationic-NLPs exhibit a zeta potential of +10 mV, although this is highly dependent on the ratio of cationic-NLP to replicon.

Cationic NLPs were formulated with RNA replicon at molar ratios ranging from 15:1 to 500:1 (NLP:RNA) ratios. As shown in FIG. 10, at increasing cationic NLP to RNA ratios, the zeta potential of the complex became more positive. Cationic NLP:RNA complexes trend toward increasingly positive zeta potential with higher NLP:RNA ratios. Non-cationic charged NLPs, such as 100% DMPC, do not alter the intrinsically negative zeta potential of the RNA replicon. Depending on the type of NLP, the transition point from negative to positive zeta potential occurred a molar ratios between 62.5:1 and 250:1 (NLP:RNA). The zeta potential distribution shown in FIG. 10 illustrates that the cationic NLP:RNA complex forms a single complex that increases in overall positive charge with increasing cationic NLP:RNA ratios.

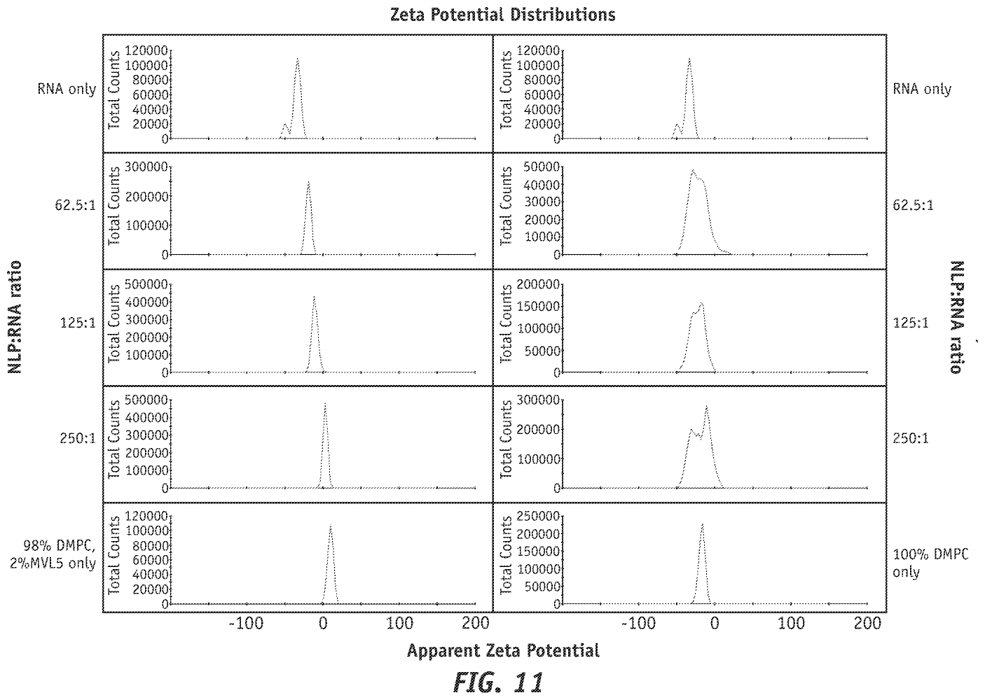

FIG. 11 shows a zeta potential distribution for a cationic NLP:Replicon complex (98:2 DMPC:MVL5, left panels) and a non-cationic NLP:Replicon mixture (100% DMPC, right panels) at the ratios of NLP:RNA indicated on the y-axis. RNA alone is shown as a control.

In the case of the non-cationic NLP:Replicon mixture (right panels), the 100% DMPC non-cationic NLPs form two distinct structures when complexed with RNA at increasing NLP:RNA ratios. The leftmost peak corresponds to the free replicon, whereas the rightmost peak is the free DMPC NLP. However, with cationic NLP:Replicon complexes, a single peak distribution was visualized throughout all the ratios tested. This phenomenon validates that cationic NLPs form stable units with RNA replicon that could not be reproduced with non-cationic charged NLPs.

### Example 5: Dynamic Light Scattering In Vitro Assessment of Cationic-NLP:Replicon Complexes

The particle size of the complex was measured using dynamic light scattering (DLS). The cationic-NLP has a size of about 13-15 nm (±1 nm, and the replicon alone has a size of about 50 nm (±10 nm. As shown in FIG. 12, the cationic-NLP:Replicon complex size varied between 90 nm and 558 nm depending on the type of cationic NLP used. When complexed with RNA, cationic NLPs form much larger structures than NLP alone. In contrast, the 100% DMPC NLP does not display a dramatic size increase when mixed with RNA.

### Example 6: Dye Exclusion In Vitro Assessment of Formation of Cationic-NLP:Replicon Complexes

An dye exclusion analysis was performed to access whether the cationic-NLP:replicon complexes protect the RNA replicon from small dye molecules. The cationic-NLP:Replicon association was assessed by measuring the level of fluorescence of cationic-NLP:Replicon after incubation with a fluorescent RNA-binding dye.

FIG. 13 shows a graph of results of dye exclusion analysis of NLP:Replicon ratio, measured in relative fluorescence units. Results are shown for NLP complexes comprising 100% DMPC (triangle), or NLP comprising DMPC with 10% DDAB (circle), or DMPC with 5% MVL5 (square).

Small dye molecules exhibit enhanced fluorescence once they intercalate between adjacent bases in the nucleic acid molecule. When associated with cationic-NLP, the RNA replicon becomes less accessible to small dye molecules, resulting in lower fluorescence. Less fluorescence was observed at higher NLP to replicon ratios. Specifically, NLPs formulated with 90:10 (DMPC:DDAB) or 95:5 (DMPC:MVL5) cationic lipids blocked most dye molecules from binding to RNA between a 250:1 and 500:1 cationic NLP:Replicon ratio. In contrast, non-cationic 100% DMPC lipids intercalated dye molecules at a relatively consistent rate despite NLP:Replicon ratio. The results suggest that cationic NLPs formulated with RNA replicon decrease dye intercalation at higher NLP:RNA ratios, whereas non-cationic NLPs, such as those comprising DMPC alone, are not affected by the NLP:RNA ratio.

### Example 7: Rnase Protection In Vitro Assessment of Cationic-NLP:Replicon Complexes

The effect of cationic-NLP association on stabilization of the RNA and protection from RNase degradation was assessed in this example. RNA molecules are formulated with increasing amounts of NLP and incubated with RNase. The amount of degradation or protection can be assessed using agarose gel electrophoresis or PCR.

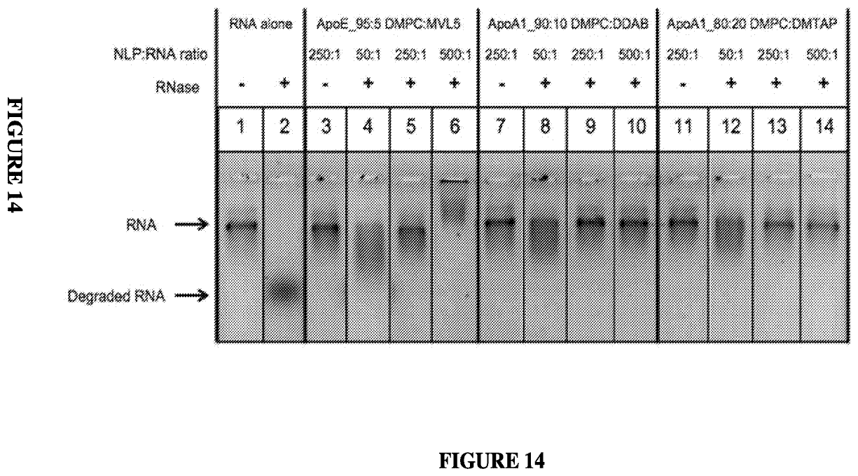

FIG. 14 shows images of gel electrophoresis results of migration patterns of RNA formulated with cationic-NLP, with (+) or without (−) RNase treatment. Each sample contains 1% triton X100 to disintegrate the NLPs and release replicon. In lane 6, the RNA migration may be affected by the highly charged cationic lipid, MVL5, which slowed RNA migration out of the well at a 500:1 ratio. 0.1 μg RNA is loaded per lane. RNA alone is shown as a control.

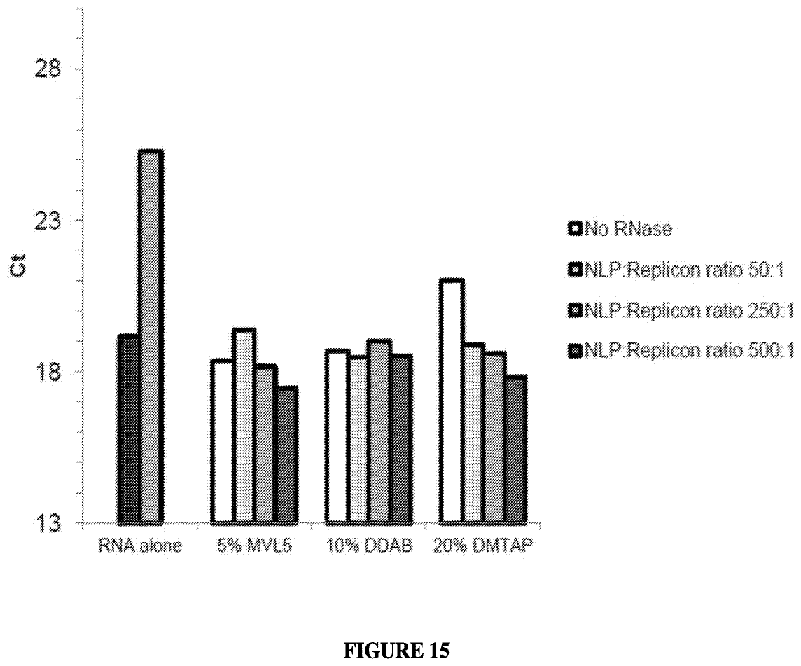

FIG. 15 shows a graph of RT-PCR cycle threshold (Ct) results for NLPs formulated with RNA replicon at varying NLP:Replicon molar ratios. As a control, RNA alone (unformulated with NLP) was treated with RNase (gray bar) or without RNase (black bar). Each sample contains 5 ng of RNA.

The results shown in FIG. 14 and FIG. 15 indicate that upon RNase treatment, the naked RNA is completely degraded. However, when RNA is complexed with cationic charged NLPs, the degradation of RNA was diminished. It is also noted that with more NLP complexed to replicon, the RNA received better protection from RNase.

### Example 8: Luciferase Activity In Vivo Assessment of Cationic-NLP:Replicon Complexes

To study the in vivo transfection efficiency of the cationic NLP:replicon complexes, a replicon with a firefly luciferase reporter gene was used. If delivered into the cytosol of a cell, this RNA molecule produces many copies of the luciferase protein. Upon injection of the luciferase substrate (luciferin), the relative expression levels of the protein in mice can be imaged in vivo using a bioluminescence imager. The amount of bioluminescence signal can be used as a surrogate for expression levels and the efficiency of the RNA delivery.

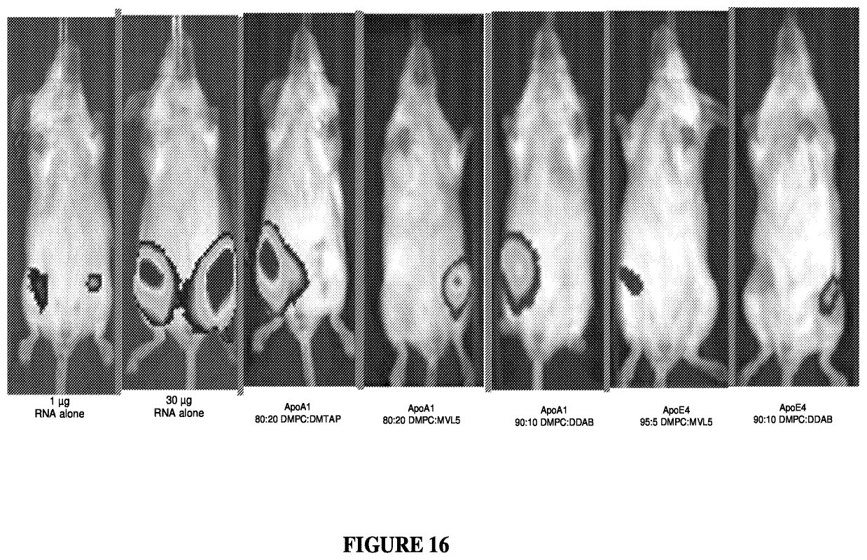

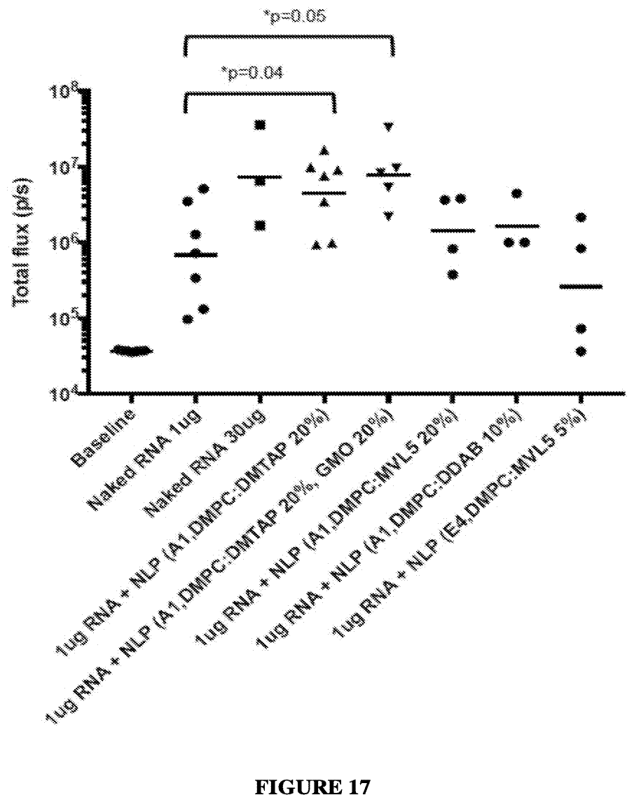

The bioluminescence was measured 4-5 days after a single bilateral i.m. injection containing a dose of 1 μg RNA replicon complexed with various NLP formulations. A bilateral i.m. injection of 1 μg and 30 μg naked RNA (unformulated with NLP) was carried out as a control. Different cationic lipid NLP formulations displayed large differences in bioluminescence intensity. Some formulations that resulted in the highest bioluminescence are shown in FIG. 16 and FIG. 17.

Mice that were injected with RNA formulated with NLPs composed of 80% DMPC bulk lipid, 20% DMTAP cationic lipid at a 250:1 (NLP:RNA) molar ratio displayed a high bioluminescence signal. In another top candidate formulation, the NLPs were formulated with 80% DMPC bulk lipid, 20% DMTAP cationic lipid, and 20% monoolein (GMO) additive at a 50:1 (NLP:RNA) molar ratio. Both of the top formulations were made with apoA1 scaffold protein. Using 1 μg of RNA, the top two NLP formulations display comparable bioluminescence to 30 μg naked RNA control. DMTAP formulations showed the strongest formulated NLP signal in vivo.

Other cationic lipid formulations also displayed relatively high bioluminescence, but luciferase signal was considerably less than the DMTAP formulations. Interestingly, NLPs formulated with 80:20 (DMPC:MVL5) and 90:10 (DMPC:DDAB) formed clean, homogeneous NLP peaks by SEC, similar to what was seen with the top DMTAP formulations. However, MVL5 and DDAB formulations did not perform as well in vivo as shown in FIG. 17.

### Example 9: Luciferase Activity In Vivo Assessment of Formation of Telo-Cationic-NLP:Replicon Complexes

Telo-cationic-NLPs can be synthesized in a similar way as the cationic-NLPs, with the exception that telodendrimer is present during the assembly process. The formulation of Telo-cationic-NLPs is similar to cationic-NLP as described in Example 1, whereby the telodendrimer is added to the assembly mixture (with the solubilized lipids and scaffold protein) prior to surfactant removal.

The effect of telodendrimer on the in vivo transfection efficiency of the cationic NLP:replicon complexes was investigated using the bioluminescence assay described in Example 8.

In particular, teledendrimer were first added to two of the top-performing cationic-NLPs formulation (apoAI DMPC:DMTAP 20% and apoAI DMPC:DMTAP 20% GMO 20%). Two different telodendrimers were explored: 5KCA8 and cys-5KCA8 (at 0.1% and 1%, respectively). Four resulting formulations were tested for the in vivo luciferase intensity, as follows:
apoAI, DMPC: 20% DMTAP+0.1% 5KCA8 telodendrimer
apoAI, DMPC: 20% DMTAP, 20% GMO+1% cys-5KCA8 telodendrimer
apoAI, DMPC: 20% DMTAP
apoAI, DMPC: 20% DMTAP, 20% GMO

The above study was duplicated to provide additional data points to increase the statistical significant of the analysis.

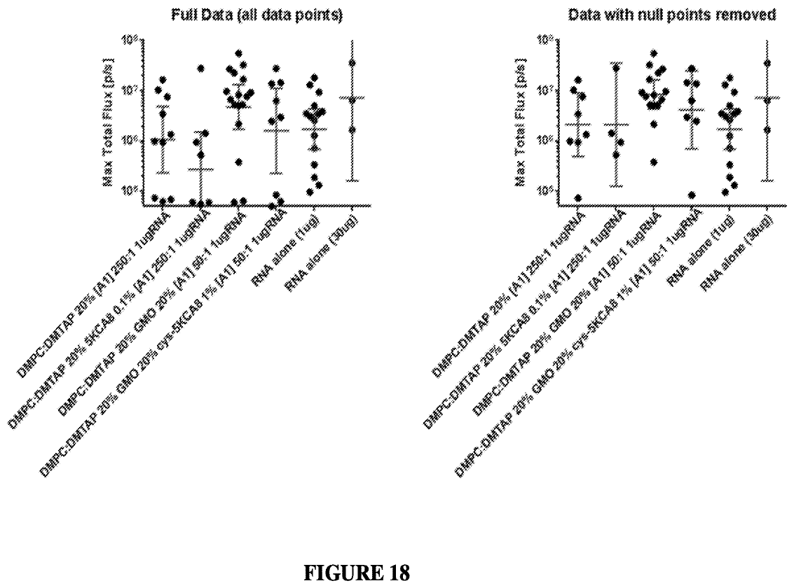

FIGS. 18 A and B summarize the in vivo luciferase intensity of RNA replicons formulated with cationic NLPs, cationic telo-NLPs, and RNA alone. Each data point represents maximal total flux from a single animal. Full data sets (left graph) contains all data points, even when no signal is detected (presumably from variability in administration). Removing null data points (right graph) illustrates effect of formulation if successfully administered. Bars indicated geometric mean with 95% confidence intervals. After initial screens, limited enhancement was observed using the telodendrimer in the two top performing cationic-NLPs, as shown in FIG. 18.

While no dramatic enhancement was observed through the addition of telodendrimer to the above NLPs formulations, it was investigated next whether adding telodendrimer to less effective cationic-NLP formulations would show greater enhancement.

Six NLP formulations from previous screens with different efficacy varying from low efficacy to high efficacy are chosen. These six NLP formulations are binned into three categories, based on their in vivo efficacy, as follows:
High efficacy group:
   apoAI, DMPC, DDAB (10%)
   apoE4, DMPC, DODAP (30%)
Medium efficacy group:
   apoE4, DMPC, MVL5 (2%)
   apoE4, DMPC, MVL5 (5%)
Low efficacy group:
   apoAI, DMPC, DMEPC (40%)
   apoAI, DMPC, DOPE (20%) DODAP (20%)

These NLPs were then formulated with the following telodendrimers at the indicated ratios:
0%, 0.1%, 0.75% 5KCA8 telodendrimer
0%, 0.1%, 0.75% 2KCA8 telodendrimer
0%, 0.1%, 0.75% cys-5KCA8 telodendrimer

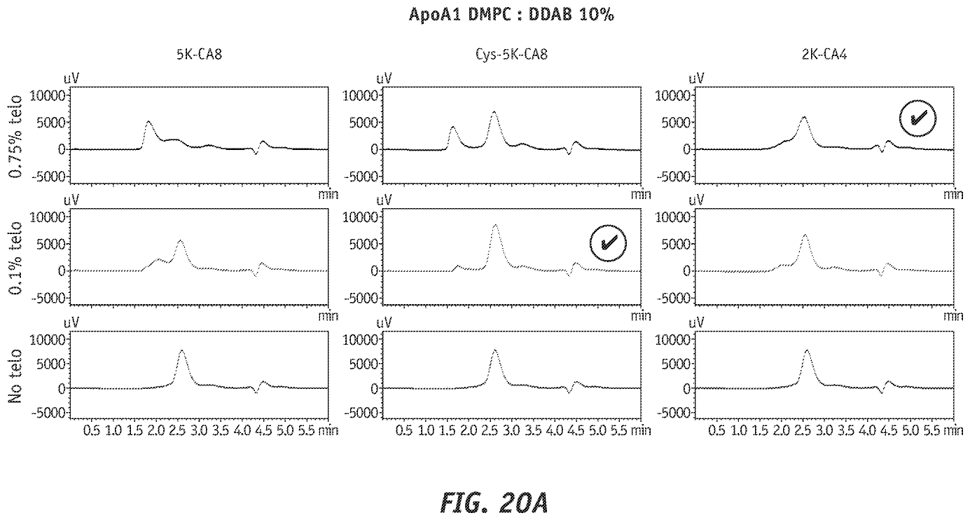

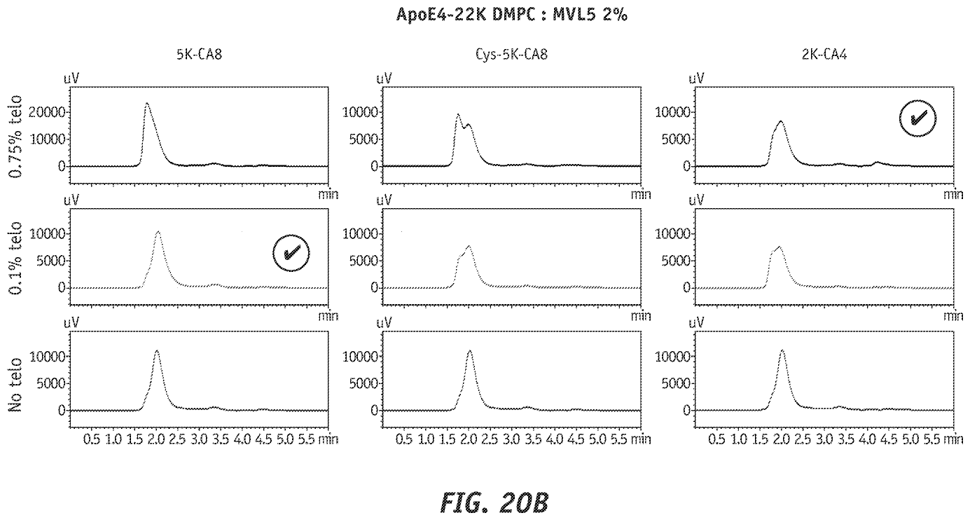

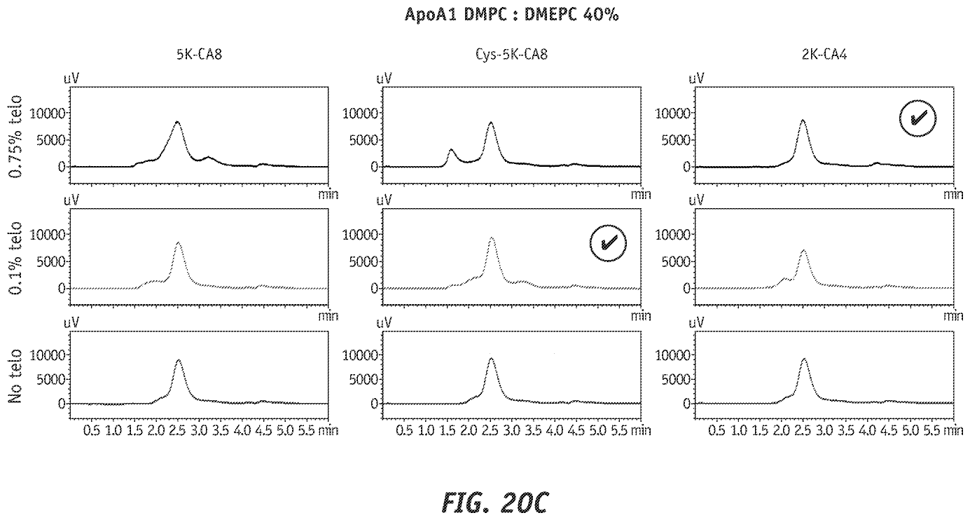

FIGS. 20A-C show the screen of NLP formation by SEC upon additional of increasing ratios of three different telodendrimers. These telodendrimers were tested with cationic NLPs binned into high efficacy (A), medium efficacy (B), or low efficacy (C), based on previous in vivo assessments of luciferase intensity. SEC traces (plotting absorbance intensity as a function of retention time) were used to identify those formulations that formed the most homogeneous species of NLPs, based on the presence of a single SEC peak. Formulations chosen for subsequent in vivo testing are marked with red check.

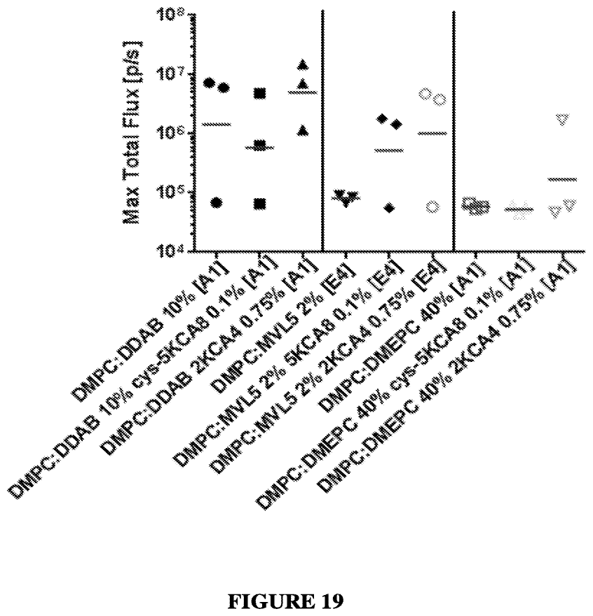

FIG. 19 summarizes in vivo luciferase intensity of RNA replicons formulated with NLPs categorized in high, medium, and low efficacy classes. No enhanced luciferase activity was observed by including telodendrimers to high efficacy NLPs (DMPC:DDAB 10% [A1]). Some enhanced luciferase activity is observed in the medium efficacy class (DMPC:MVL5 2% [E4]) upon the addition of either 5KCA8 (0.1%) or 2KCA4 (0.75%) telodendrimer. No significant enhancement in luciferase activity is observed in the low efficacy class (DMPC:DMEPC 40% [E4]) upon the addition of telodendrimer. Each data point represents maximal total flux from a single animal. Bars indicated geometric mean.

The results suggest that addition of telodendrimer appears to enhance luciferase signal compared to non-telo formulations. The overall effect is telo- and NLP-dependent. The greatest improvement was with the medium efficacy group, where only telodendrimer-based formulations elicited significant luciferase activity.

The examples set forth above are provided to give those of ordinary skill in the art a complete disclosure and description of how to make and use the embodiments of the materials, compositions, systems and methods of the disclosure, and are not intended to limit the scope of what the inventors regard as their disclosure. Those skilled in the art will recognize how to adapt the features of the exemplified NLPs and related uses to additional NLPs formed by other cationic lipids, membrane forming lipids, scaffold proteins, additives, and possibly functionalized amphipathic compounds and membrane proteins according to various embodiments and scope of the claims.

All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified may be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein may be excluded from a claim of this disclosure. The disclosure illustratively described herein suitably may be practiced in the absence of any element or elements, limitation or limitations which is not specifically disclosed herein.

A number of embodiments of the disclosure have been described. The specific embodiments provided herein are examples of useful embodiments of the invention and it will be apparent to one skilled in the art that the disclosure can be carried out using a large number of variations of the devices, device components, methods steps set forth in the present description. As will be obvious to one of skill in the art, methods and devices useful for the present methods may include a large number of optional composition and processing elements and steps.

In particular, it will be understood that various modifications may be made without departing from the spirit and scope of the present disclosure. Accordingly, other embodiments are within the scope of the following claims.

Although the invention has been described with reference to the above examples, it will be understood that modifications and variations are encompassed within the spirit and scope of the invention. Accordingly, the invention is limited only by the following claims.

### REFERENCES

1. Fischer, N. O., et al., Evaluation of Nanolipoprotein Particles (NLPs) as an In Vivo Delivery Platform. PLoS ONE, 2014. 9(3): p. e93342.
2. Blanchette, C. D., et al., Kinetic Analysis of His-Tagged Protein Binding to Nickel-Chelating Nanolipoprotein Particles. Bioconjugate Chemistry, 2010. 21(7): p. 1321-1330.
3. Fischer, N. O., et al., Colocalized Delivery of Adjuvant and Antigen Using Nanolipoprotein Particles Enhances the Immune Response to Recombinant Antigens. Journal of the American Chemical Society, 2013. 135(6): p. 2044-2047.
4. Shih, A. Y., et al., Disassembly of nanodiscs with cholate. Nano Letters, 2007. 7(6): p. 1692-1696.
5. Chromy, B. A., et al., Different apolipoproteins impact nanolipoprotein particle formation. Journal of the American Chemical Society, 2007. 129(46): p. 14348-14354.
6. Blanchette, C. D., et al., Characterization and Purification of Polydisperse Reconstituted Lipoproteins and Nanolipoprotein Particles. International Journal of Molecular Sciences, 2009. 10(7): p. 2958-2971.
7. CHROMY, B. A., NANOLIPOPROTEIN PARTICLES AND RELATED METHODS AND SYSTEMS FOR PROTEIN CAPTURE, SOLUBILIZATION, AND/OR PURIFICATION in US 2009-0192299 A1 2009: US.
8. Cappuccio, J. A., et al., Cell-free Co-expression of Functional Membrane Proteins and Apolipoprotein, Forming Soluble Nanolipoprotein Particles. Molecular & Cellular Proteomics, 2008. 7(11): p. 2246-2253.
9. Gao, T. J., et al., Characterizing diffusion dynamics of a membrane protein associated with nanolipoproteins using fluorescence correlation spectroscopy. Protein Science, 2011. 20(2): p. 437-447.
10. Katzen, F., et al., Insertion of membrane proteins into discoidal membranes using a cell-free protein expression approach. Journal of Proteome Research, 2008. 7(8): p. 3535-3542.
11. Coleman, M. A., METHODS AND SYSTEMS FOR PRODUCING NANOLIPOPROTEIN PARTICLES, in US 2011-0059549 A1 2008: US.
12. Georger, J. H., et al., Helical and Tubular Microstructures Formed by Polymerizable Phosphatidylcholines. J Am Chem Soc, 1987. 109(20): p. 6169-6175.
13. Morigaki, K., et al., Surface Functionalization of a Polymeric Lipid Bilayer for Coupling a Model Biological Membrane with Molecules, Cells, and Microstructures. Langmuir, 2013. 29(8): p. 2722-2730.
14. Regen, S. L., et al., Polymerized Phosphatidyl Choline Vesicles—Stabilized and Controllable Time-Release Carriers. Biochemical and Biophysical Research Communications, 1981. 101(1): p. 131-136.
15. Bolikal, D. and S. L. Regen, Degree of Polymerization of a Vesicle Membrane. Macromolecules, 1984. 17(6): p. 1287-1289.
16. Lei, J. T. and D. F. Obrien, 2-Dimensional Polymerization of Lipid Bilayers—Rate of Polymerization of Acryloyl and Methacryloyl Lipids. Macromolecules, 1994. 27(6): p. 1381-1388.
17. Sells, T. D. and D. F. Obrien, 2-Dimensional Polymerization of Lipid Bilayers—Degree of Polymerization ofAcryloyl Lipids. Macromolecules, 1994. 27(1): p. 226-233.
18. Lamparski, H. and D. F. Obrien, 2-Dimensional Polymerization of Lipid Bilayers—Degree of Polymerization of Sorbyl Lipids. Macromolecules, 1995. 28(6): p. 1786-1794.
19. Tsuchida, E., et al., Polymerization of Unsaturated Phospholipids as Large Unilamellar Liposomes at Low-Temperature. Macromolecules, 1992. 25(1): p. 207-212.
20. Ohno, H., Y. Ogata, and E. Tsuchida, Polymerization of Liposomes Composed of Diene-Containing Lipids by Uv and Radical Initiators—Evidence for the Different Chemical Environment of Diene Groups on 1-Acyl and 2-Acyl Chains. Macromolecules, 1987. 20(5): p. 929-933.
21. Serrano, J., et al., Polymerized Surfactant Vesicles—Determinations of Rates and Degrees of Polymerization in Vesicles Prepared from Styrene-Containing Surfactants. Macromolecules, 1985. 18(10): p. 1999-2005.
22. Lieser, G., B. Tieke, and G. Wegner, Structure, Phase-Transitions and Polymerizability of Multilayers of Some Diacetylene Monocarboxylic Acids. Thin Solid Films, 1980. 68(1): p. 77-90.
23. Johnston, D. S., et al., Phospholipid polymers—synthesis and spectral characteristics. Biochim Biophys Acta, 1980. 602(1): p. 57-69.
24. Kim, J. M., et al., Immobilized polydiacetylene vesicles on solid substrates for use as chemosensors. Advanced Materials, 2003. 15(13): p. 1118-1121.
25. Hayward, J. A. and D. Chapman, Biomembrane surfaces as models for polymer design: the potential for haemocompatibility. Biomaterials, 1984. 5(3): p. 135-42.
26. Regen, S. L., et al., Polymerized phosphatidyl choline vesicles. Stabilized and controllable time-release carriers. Biochem Biophys Res Commun, 1981. 101(1): p. 131-6.
27. Yavlovich, A., et al., A novel class of photo-triggerable liposomes containing DPPC:DC8,9PC as vehicles for delivery of doxorubcin to cells. Biochimica Et Biophysica Acta-Biomembranes, 2011. 1808(1): p. 117-126.
28. Tieke, B., G. Lieser, and G. Wegner, Polymerization of diacetylenes in multilayers. Journal of Polymer Science: Polymer Chemistry Edition, 1979. 17(6): p. 1631-1644.
29. Baughman, R. H., Solid-state polymerization of diacetylenes. Journal of Applied Physics, 1972. 43(11): p. 4362-4370.
30. Weilhammer, D. R., et al., The use of nanolipoprotein particles to enhance the immunostimulatory properties of innate immune agonists against lethal influenza challenge. Biomaterials, 2013. 34(38): p. 10305-18.
31. Wang, S., D. Q. Peng, and Y. Yi, The unsolved mystery of apoA-I recycling in adipocyte. Lipids Health Dis, 2016. 15: p. 35.
32. Vickers, K. C., et al., MicroRNAs are transported in plasma and delivered to recipient cells by high-density lipoproteins. Nat Cell Biol, 2011. 13(4): p. 423-33.
33. Coleman, M. A., et al., Expression and Association of the Yersinia pestis Translocon Proteins, YopB and YopD, Are Facilitated by Nanolipoprotein Particles. PLoS One, 2016. 11(3): p. e0150166.
34. He, W., et al., Cell-free expression of functional receptor tyrosine kinases. Sci Rep, 2015. 5: p. 12896.
35. Tufteland, M., G. Ren, and R. Ryan, Nanodisks derivedfrom amphotericin B lipid complex. Journal of Pharmaceutical Sciences, 2008. 97(10): p. 4425-4432.
36. Yuan, Y., et al., Delivery of hydrophilic drug doxorubicin hydrochloride-targeted liver using apoAI as carrier. J Drug Target, 2013. 21(4): p. 367-374.
37. Ding, Y., et al., A biomimetic nanovector-mediated targeted cholesterol-conjugated siRNA delivery for tumor gene therapy. Biomaterials, 2012. 33(34): p. 8893-8905.
38. Frias, J. C., et al., Properties of a versatile nanoparticle platform contrast agent to image and characterize atherosclerotic plaques by magnetic resonance imaging. Nano Lett, 2006. 6(10): p. 2220-2224.
39. Fischer, N. O., et al., Conjugation to nickel-chelating nanolipoprotein particles increases the potency and efficacy of subunit vaccines to prevent West Nile encephalitis. Bioconjug Chem, 2010. 21(6): p. 1018-22.
40. Fischer, N. O., et al., Colocalized delivery of adjuvant and antigen using nanolipoprotein particles enhances the immune response to recombinant antigens. J Am Chem Soc, 2013. 135(6): p. 2044-7.
41. Bhattacharya, P., et al., Nanodisc-Incorporated Hemagglutinin Provides Protective Immunity against Influenza Virus Infection. Journal of Virology, 2010. 84(1): p. 361-371.
42. Allen, T. M. and P. R. Cullis, Drug delivery systems: entering the mainstream. Science, 2004. 303(5665): p. 1818-22.
43. Sparreboom, A., et al., Comparative preclinical and clinical pharmacokinetics of a cremophor-free, nanoparticle albumin-bound paclitaxel (ABI-007) and paclitaxel formulated in Cremophor (Taxol). Clin Cancer Res, 2005. 11(11): p. 4136-43.
44. Cho, K., et al., Therapeutic nanoparticles for drug delivery in cancer. Clinical cancer research, 2008. 14(5): p. 1310-1316.
45. al., A. G. C. e., Emerging implications of nanotechnology on cancer diagnostics and therapeutics. Cancer, 2006. 107: p. 8.
46. al., S. M. e., A concise review of carbon nanotube's toxicology. Nano Rev., 2013. 4.
47. Miyazaki, M., et al., Effect of phospholipid composition on discoidal HDL formation. Biochimica et Biophysica Acta (BBA)-Biomembranes, 2013. 1828(5): p. 1340-1346.
48. Weilhammer, D. R., et al., The use of nanolipoprotein particles to enhance the immunostimulatory properties of innate immune agonists against lethal influenza challenge. Biomaterials, 2013. 34(38): p. 10305-10318.
49. Cappuccio, J. A., et al., Cell-free co-expression of functional membrane proteins and apolipoprotein, forming soluble nanolipoprotein particles. Molecular & Cellular Proteomics, 2008. 7(11): p. 2246-2253.
50. Cappuccio, J. A., et al., Cell-free expression for nanolipoprotein particles: building a high-throughput membrane protein solubility platform, in High throughput protein expression and purification. 2009, Springer. p. 273-295.
51. Blanchette, C. D., et al., Atomic force microscopy differentiates discrete size distributions between membrane protein containing and empty nanolipoprotein particles. Biochimica et Biophysica Acta (BBA)-Biomembranes, 2009. 1788(3): p. 724-731.
52. Wadsdter, M., et al., Monitoring shifts in the conformation equilibrium of the membrane protein cytochrome P450 reductase (POR) in nanodiscs. Journal of Biological Chemistry, 2012. 287(41): p. 34596-34603.
53. Justesen, B. H., et al., Isolation of monodisperse nanodisc-reconstituted membrane proteins using free flow electrophoresis. Analytical chemistry, 2013. 85(7): p. 3497-3500.
54. Baylon, J. L., et al., Characterizing the membrane-bound state of cytochrome P450 3A4: structure, depth of insertion, and orientation. Journal of the American Chemical Society, 2013. 135(23): p. 8542-8551.
55. Gao, T., et al., Characterization of De Novo Synthesized GPCRs Supported in Nanolipoprotein Discs. PLoS ONE, 2012. 7(9): p. e44911.
56. Akkaladevi, N., et al., Assembly of anthrax toxin pore: Lethal-factor complexes into lipid nanodiscs. Protein Science, 2013. 22(4): p. 492-501.
57. Tufteland, M., et al., Peptide stabilized amphotericin B nanodisks. Peptides, 2007. 28(4): p. 741-746.
58. Jia, J., et al., Preparation, characterizations, and in vitro metabolic processes of paclitaxel-loaded discoidal recombinant high-density lipoproteins. Journal of Pharmaceutical Sciences, 2012. 101(8): p. 2900-2908.
59. Wang, J., et al., Tumor targeting effects of a novel modified paclitaxel-loaded discoidal mimic high density lipoproteins. Drug delivery, 2013. 20(8): p. 356-363.
60. Fischer, N. O., et al., Conjugation to Nickel-Chelating Nanolipoprotein Particles Increases the Potency and Efficacy of Subunit Vaccines to Prevent West Nile Encephalitis. Bioconjugate Chemistry, 2010. 21(6): p. 1018-1022.
61. Rensen, P. C. N., et al., Recombinant lipoproteins: lipoprotein-like lipid particles for drug targeting. Advanced Drug Delivery Reviews, 2001. 47(2-3): p. 251-276.
62. Blanchette, C. D., et al., Quantifying size distributions of nanolipoprotein particles with single-particle analysis and molecular dynamic simulations. Journal of Lipid Research, 2008. 49(7): p. 1420-1430.

1. A nanolipoprotein particle comprising:
a membrane forming lipid, a cationic lipid and a scaffold protein,
wherein the membrane forming lipid and the cationic lipid are arranged in a membrane forming lipid bilayer stabilized by the scaffold protein.

2. The nanolipoprotein particle of claim 1, wherein the cationic lipid is in a molar concentration of about 1 to about 60 mol %.

3. The nanolipoprotein particle of claim 1, wherein the cationic lipid is in molar concentration of about 5 to about 40 mol %.

4. The nanolipoprotein particle of claim 1, wherein a total lipid to scaffold protein molar percent ratio ranges from 20:1 to 240:1.

5. The nanolipoprotein particle of claim 4, wherein the total lipid to scaffold protein molar percent ratio is 80:1.

6. The nanolipoprotein particle of claim 1, wherein the cationic lipid comprises at least one cationic lipid selected from the group consisting of dimethyldioctadecylammonium (DDAB), 1,2-dimyristoleoyl-sn-glycero-3-ethylphosphocholine (DMEPC), 1,2-dimyristoyl-3-trimethylammonium-propane (DMTAP), 1,2-dioleoyl-3-dimethylammonium-propane (DODAP), 1,2-dioleoyl-sn-glycero-3-ethylphosphocholine (DOEPC), dioleoylphosphoethanolamine (DOPE), 1,2-dioleoyl-3-trimethylammonium-propane (DOTAP), 1,2-di-O-octadecenyl-3-trimethylammonium propane (DOTMA) and N1-[2-((1S)-1-[(3-aminopropyl)amino]-4-[di(3-amino-propyl)amino]butylcarboxamido)ethyl]-3,4-di[oleyloxy]-benzamide (MVL5).

7. The nanolipoprotein particle of claim 1, wherein the cationic lipid comprise lipids of Formula (I) 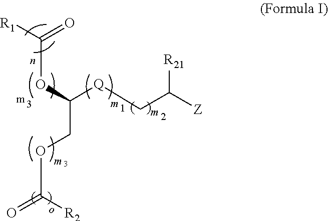
wherein
R1 and R2 are independently selected from H and a C7-C29 branched or straight, substituted or unsubstituted aliphatic carbon chain;
R₂₁ is H, OH, or a carboxy group;
Q is selected from: 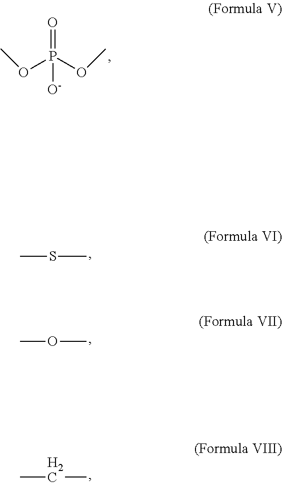
m₁=0-1; m₂=0-3; m₃=0-1 and
n and o are independently 0 and 1;
Z is a moiety of formula (II) or Formula (III) or Formula (IV), wherein
the moiety of Formula (II) is 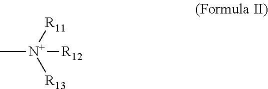
in which R₁₁, R₁₂ and R₁₃ are independently H or a C1-C4 branched or straight aliphatic carbon chain;
the moiety of Formula (III) is 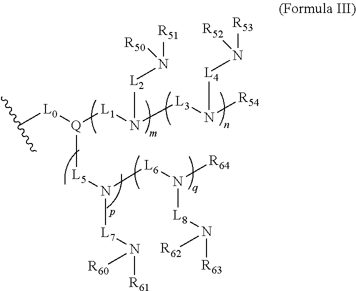
wherein
Q is N or CH;
m, n, p and q are independently 0-3 and m+n+p+q is equal or greater than 1;
L₀-L₈ are independently a divalent group selected from C₁₋₆ alkylidenyl, C₁₋₆ heteroalkylidenyl, C₃₋₆ cycloalkylidenyl, and C₃₋₆ heterocycloalkylidenyl, wherein each of the alkylidenyl, heteroalkylidenyl, cycloalkylidenyl, and heterocycloalkylidenyl is optionally substituted with one or more substituents independently selected from C₁₋₄ alkyl, haloalkyl, C₁₋₄ alkoxy, amino, alkylamino, dialkylamino, oxo groups;
R₅₀-R₅₄, R₆₀-R₆₄ are independently a monovalent group selected from H, C₁₋₆ alkyl, C₁₋₆ heteroalkyl, C₃₋₆ cycloalkyl, and C₃₋₆ heterocycloalkyl, wherein each of the alkyl, heteroalkyl, cycloalkyl, and heterocycloalkyl is optionally substituted with one or more substituents independently selected from C₁₋₄ alkyl, haloalkyl, C₁₋₄ alkoxy, amino, C₁₋₄ alkylamino, C₁₋₄ dialkylamino, or a oxo group; and
the moiety of Formula (IV) is 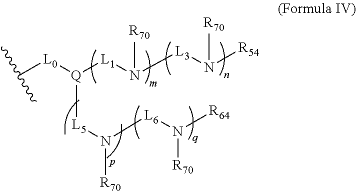
in which Q is N or CH;
m, n, p and q are independently 0-3 and m+n+p+q is equal or greater than 1;
L₀-L₁; L₃; and L₅-L₆ are independently a divalent group selected from C₁₋₆ alkylidenyl, C₁₋₆ heteroalkylidenyl, C₃₋₆ cycloalkylidenyl, and C₃₋₆ heterocycloalkylidenyl, wherein each of the alkylidenyl, heteroalkylidenyl, cycloalkylidenyl, and heterocycloalkylidenyl is optionally substituted with one or more substituents independently selected from C₁₋₄ alkyl, haloalkyl, C₁₋₄ alkoxy, amino, alkylamino, dialkylamino, oxo groups;
R₅₄, R₆₄ and R₇₀ are independently a monovalent group selected from H, C₁₋₆ alkyl, C₁₋₆ heteroalkyl, C₃₋₆ cycloalkyl, and C₃₋₆ heterocycloalkyl, wherein each of the alkyl, heteroalkyl, cycloalkyl, and heterocycloalkyl is optionally substituted with one or more substituents independently selected from C₁₋₄ alkyl, haloalkyl, C₁₋₄ alkoxy, amino, C₁₋₄ alkylamino, C₁₋₄ dialkylamino, or a oxo group.

8. The nanolipoprotein particle of claim 1, wherein the membrane forming lipid, cationic lipid and the scaffold protein are at a ratio from 19:1:1 to 96:144:1.

9. The nanolipoprotein particle of claim 1, wherein the membrane forming lipid comprises dimyristoylphosphatidylcholine (DMPC) and the cationic lipid comprises DMTAP.

10. The nanolipoprotein particle of claim 9, wherein the DMPC and DMTAP is at a ratio about 8:2.

11. The nanolipoprotein particle of claim 1, wherein the scaffold protein is selected from the group comprising apolipoproteins A (apo A-I, apo A-II, apo A-IV, and apo A-V), apolipoproteins B (apo B48 and apo B100), apolipoproteins C (apo C-I, apo C-II, apo C-III, and apo C-IV), apolipoproteins D, apolipoproteins E, apolipoproteins H, apolipoprotein E4 (22 Kd fragment), lipophorin III, apolipoprotein A-1 and any derivative or a fragment thereof.

12. The nanolipoprotein particle of claim 1, wherein the scaffold protein is apoA1, the membrane forming lipid is DMPC and the cationic lipid is DDAB, the DMPC and DDAB at a ratio about 9:1.

13. The nanolipoprotein particle of claim 1, wherein the scaffold protein is apoA1, the membrane forming lipid is DMPC and the cationic lipid is DDAB, the DMPC and DDAP, at a ratio about 7:3.

14. The nanolipoprotein particle of claim 1, wherein the scaffold protein is apoE4, the membrane forming lipid is DMPC and the cationic lipid is DODAP, the DMPC and DODAP, at a ratio about 7:3.

15. The nanolipoprotein particle of claim 1, wherein the scaffold protein is apoE4, the membrane forming lipid is DMPC and the cationic lipid is MVL5.

16. The nanolipoprotein particle of claim 1, further comprising one or more additives selected from the group consisting of cholesterol, CpG-cholesterol, lyso lipids, monoolein (GMO), Tween 20 and Z3-14.

17. The nanolipoprotein particle of claim 16, wherein the molar ratio of one or more additives to total lipid content is about 1:99 to 3:2.

18. The nanolipoprotein particle of claim 16, wherein the membrane forming lipid is DMPC, the cationic lipid is DMTAP, and the one or more additives is GMO, at a ratio of about 8:2:2.

19. The nanolipoprotein particle of claim 1, further comprising one or more telodendrimers.

20. The nanolipoprotein particle of claim 19, wherein the telodendrimers are at a ratio of about 0.1% to 10% of a total lipid component of the membrane forming lipid and the cationic lipid.

21.-52. (canceled)
